SEQUENCE LISTING (50 sequences)

SEQ ID NO: 1 - DNA, 780 bp, Artificial Sequence
  Synthetic protein
  CDS (1)..(780)
atgctgatcagcctgctggaggtgatcgagcccgaggtgctgtacagcggctacgacagcaccctgcccgacaccagcaccaggctgatgagcaccctgaacaggctgggcggcaggcaggtggtgagcgccgtgaagtgggccaaggccctgcccggcttcaggaacctgcacctggacgaccagatgaccctgctgcagtacagctggatgagcctgatggccttcagcctgggctggaggagctacaagcagagcaacggcaacatgctgtgcttcgcccccgacctggtgatcaacgaggagaggatgcagctgccctacatgtacgaccagtgccagcagatgctgaagatcagcagcgagttcgtgaggctgcaggtgagctacgacgagtacctgtgcatgaaggtgctgctgctgctgagcaccgtgcccaaggacggcctgaagagccaggccgtgttcgacgagatcaggatgacctacatcaaggagctgggcaaggccatcgtgaagagggagggcggcggctctggcggcggctccggaggctctaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattctggttctggatacccatacgatgttccagattacgcttaa

SEQ ID NO: 2 - protein, 259 aa, Artificial Sequence
  Synthetic Protein
MLISLLEVIEPEVLYSGYDSTLPDTSTRLMSTLNRLGGRQVVSAVKWAKALPGFRNLHLDDQMTLLQYSWMSLMAFSLGWRSYKQSNGNMLCFAPDLVINEERMQLPYMYDQCQQMLKISSEFVRLQVSYDEYLCMKVLLLLSTVPKDGLKSQAVFDEIRMTYIKELGKAIVKREGGGSGGGSGGSTMTEKEIVDYVASQVTTAKKLRGGVVFVDEVPKGLTGKLDARKIREILIKAKKGGKIAVNSGSGYPYDVPDYA

SEQ ID NO: 3 - DNA, 1758 bp, Artificial Sequence
  Synthetic protein
  CDS (1)..(1758)
atggagcagaagctgatcagcgaggaggacctgggttctggagaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaggcggctctggcggcggctccggaggctctaacagcagccagaactggcagaggttctaccagctgaccaagctgctggacagcatgcacgagatggtgggcggcctgctccagttctgcttctacaccttcgtgaacaagagcctgagcgtggagttccccgagatgctggccgagatcatcagcaaccagctgcccaagttcaaggccggcagcgtgaagcccctgctgttccaccagaagtaa

SEQ ID NO: 4 - protein, 585 aa, Artificial Sequence
  Synthetic Protein
MEQKLISEEDLGSGEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKGGSGGGSGGSNSSQNWQRFYQLTKLLDSMHEMVGGLLQFCFYTFVNKSLSVEFPEMLAEIISNQLPKFKAGSVKPLLFHQK

SEQ ID NO: 5 - DNA, 813 bp, Artificial Sequence
  Synthetic protein
  CDS (1)..(813)
atggacgctctgtcacctgaacagctcgttttgacgcttttggaagctgaaccaccacacgttctgatcagccgaccctctgctccattcacagaggccagtatgatgatgagcctcactaagttggccgataaagaacttgtccatatgatttcctgggcgaagaaaatacccggattcgtagagctttccctcttcgatcaggtacggctgctcgagagttgctggatggaggttctgatgatggggctgatgtggagatccatagatcaccccggtaaacttatctttgcgccagaccttgtactcgaccgggacgaaggaaaatgcgtagaagggatcctggaaatttttgatatgctgctcgctaccaccagtcggttcagggagctgaaattgcagcacaaggaatatctttgcgtgaaagcgatgatacttctcaattcaagtatgtaccccctcgtgacagcaacccaggacgcagatagctccagaaaacttgctcatcttcttaatgcggtcaccgacgcattggtctgggttattgcaaagtctggtataggcggctctggcggcggctccggaggctctaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattctggttctggatacccatacgatgttccagattacgcttaa

SEQ ID NO: 6 - protein, 270 aa, Artificial Sequence
  Synthetic Protein
MDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGIGGSGGGSGGSTMTEKEIVDYVASQVTTAKKLRGGVVFVDEVPKGLTGKLDARKIREILIKAKKGGKIAVNSGSGYPYDVPDYA

SEQ ID NO: 7 - DNA, 1710 bp, Artificial Sequence
  Synthetic protein
  CDS (1)..(1710)
atggagcagaagctgatcagcgaggaggacctgggttctggagaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaggcggctctggcggcggctccggaggctcttccagccagcaacaatctatgcgattggcgaacttgcttatgctgctcagtcacgtaaggcatgccagcaataaagggatggaacatcttcttaatatgaagtgtaagaatgtggtgcccgtttacgatctcctgctggaaatgctcaacgcacacgttctcaggtaa

SEQ ID NO: 8 - protein, 569 aa, Artificial Sequence
  Synthetic Protein
MEQKLISEEDLGSGEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKGGSGGGSGGSSSQQQSMRLANLLMLLSHVRHASNKGMEHLLNMKCKNVVPVYDLLLEMLNAHVLR

SEQ ID NO: 9 - DNA, 894 bp, Artificial Sequence
  Synthetic protein
  CDS (1)..(894)
atgcacatggaggagctgcagaagagcatcggccacaagcccgagcccaccgacgaggagtgggagctgatcaagaccgtgaccgaggcccacgtggccaccaacgcccagggcagccactggaagcagaagaggaagttcctgcccgaggacatcggccaggcccccatcgtgaacgcccccgagggcggcaaggtggacctggaggccttcagccacttcaccaagatcatcacccccgccatcaccagggtggtggacttcgccaagaagctgcccatgttctgcgagctgccctgcgaggaccagatcatcctgctgaagggctgctgcatggagatcatgagcctgagggccgccgtgaggtacgaccccgagagcgagaccctgaccctgaacggcgagatggccgtgaccaggggccagctgaagaacggcggcctgggcgtggtgagcgacgccatcttcgacctgggcatgagcctgagcagcttcaacctggacgacaccgaggtggccctgctgcaggccgtgctgctgatgagcagcgacaggcccggcctggcctgcgtggagaggatcgagaagtaccaggacagcttcctgctggccttcgagcactacatcaactacaggaagcacggcggctctggcggcggctccggaggctctaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattctggttctggatacccatacgatgttccagattacgcttaa

SEQ ID NO: 10 - protein, 297 aa, Artificial Sequence
  Synthetic Protein
MHMEELQKSIGHKPEPTDEEWELIKTVTEAHVATNAQGSHWKQKRKFLPEDIGQAPIVNAPEGGKVDLEAFSHFTKIITPAITRVVDFAKKLPMFCELPCEDQIILLKGCCMEIMSLRAAVRYDPESETLTLNGEMAVTRGQLKNGGLGVVSDAIFDLGMSLSSFNLDDTEVALLQAVLLMSSDRPGLACVERIEKYQDSFLLAFEHYINYRKHGGSGGGSGGSTMTEKEIVDYVASQVTTAKKLRGGVVFVDEVPKGLTGKLDARKIREILIKAKKGGKIAVNSGSGYPYDVPDYA

SEQ ID NO: 11 - DNA, 1689 bp, Artificial Sequence
  Synthetic Protein
  CDS (1)..(1689)
atggagcagaagctgatcagcgaggaggacctgggttctggagaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaggcggctctggcggcggctccggaggctctcacgtgacccacttctggcccaagctgctgatgaaggtgaccgacctgaggatgatcggcgcctgccacgccagcaggttcctgcacatgaaggtggagtgccccaccgagctgttcccccccctgttcctggaggtgttcgagtaa

SEQ ID NO: 12 - protein, 562 aa, Artificial Sequence
  Synthetic Protein
MEQKLISEEDLGSGEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKGGSGGGSGGSHVTHFWPKLLMKVTDLRMIGACHASRFLHMKVECPTELFPPLFLEVFE

SEQ ID NO: 13 - DNA, 945 bp, Artificial Sequence
  Synthetic Protein
  CDS (1)..(945)
atgggcagccacatgttgaatcctgaaagcgctgatttgcgcgcacttgctaaacatctctacgattcttatatcaaatcattccccttgaccaaagcaaaagcgagagctatacttaccggtaagacaacggataagtccccattcgttatttatgacatgaattcacttatgatgggtgaagataagataaaattcaaacatataactcctctccaagagcaatcaaaggaagttgcgattagaatcttccagggctgtcaattcagaagcgtagaagcggtccaggagattacggagtatgcaaaatctatccccggctttgtcaacctcgaccttaacgaccaagtcactcttcttaaatacggggtacacgaaattatatacaccatgcttgcgtccctgatgaacaaagacggtgtgctgattagtgaaggtcaggggtttatgacgcgggaattccttaagagtcttagaaaacctttcggggactttatggagccaaagttcgagtttgcagtgaaatttaatgcactggaactcgacgactctgacctggctatattcatagccgttattattctcagcggagaccggcctggactcctcaacgtgaagccgattgaggacatacaggataacctgctccaagcccttgagctgcaactgaagctcaatcaccccgaatctagcggcggctctggcggcggctccggaggctctaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattctggttctggatacccatacgatgttccagattacgcttaa

SEQ ID NO: 14 - protein, 314 aa, Artificial Sequence
  Synthetic Protein
MGSHMLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSGGSGGGSGGSTMTEKEIVDYVASQVTTAKKLRGGVVFVDEVPKGLTGKLDARKIREILIKAKKGGKIAVNSGSGYPYDVPDYA

SEQ ID NO: 15 - DNA, 1689 bp, Artificial Sequence
  Synthetic Protein
  CDS (1)..(1689)
atggagcagaagctgatcagcgaggaggacctgggttctggagaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaggcggctctggcggcggctccggaggctctcagctctttgccaagttgctccagaagatgactgacctgcgacaaatagtgacagagcacgtccaactgctccaagtgatcaaaaagaccgaaacggatatgagtcttcaccccctgttgcaggaaatatacaaggacctgtactaa

SEQ ID NO: 16 - protein, 562 aa, Artificial Sequence
  Synthetic Protein
MEQKLISEEDLGSGEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKGGSGGGSGGSQLFAKLLQKMTDLRQIVTEHVQLLQVIKKTETDMSLHPLLQEIYKDLY

SEQ ID NO: 17 - DNA, 342 bp, Artificial Sequence
  Synthetic protein
  CDS (1)..(342)
atggcctgggagatcccccgggagtccatcaagttggtgaaaaggcttggcgctgggcagtttggggaagtctggatgggttactataacggcggctctggcggcggctccggaggctctaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattctggttctggatacccatacgatgttccagattacgcttaa

SEQ ID NO: 18 - protein, 113 aa, Artificial Sequence
  Synthetic Protein
MAWEIPRESIKLVKRLGAGQFGEVWMGYYNGGSGGGSGGSTMTEKEIVDYVASQVTTAKKLRGGVVFVDEVPKGLTGKLDARKIREILIKAKKGGKIAVNSGSGYPYDVPDYA

SEQ ID NO: 19 - DNA, 2280 bp, Artificial Sequence
  Synthetic protein
  CDS (1)..(2280)
atggagcagaagctgatcagcgaggaggacctgggttctggagaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaggcggctctggcggcggctccggaggctctaacagtaccaaggtggctgtgaaaaccctgaagccaggaactatgtctgtgcaagccttcctggaagaagccaacctcatgaagaccctgcagcatgacaagctcgtgaggctctacgctgtggtcaccagggaggagcccatttacatcatcaccgagtacatggccaagggcagtttgctggatttcctgaagagcgatgaaggtggcaaagtgctgcttccaaagctcattgacttttctgctcagattgcagagggaatggcatacatcgagcggaagaactacattcaccgggacctgcgagcagctaatgttctggtctccgagtcactcatgtgcaaaattgcagattttggccttgctagagtaattgaagataatgagtacacagcaagggaaggtgctaagttccctattaagtggacggctccagaagcaatcaactttggatgtttcactattaagtctgatgtgtggtcctttggaatcctcctatacgaaattgtcacctatgggaaaattccctacccagggagaactaatgccgacgtgatgaccgccctgtcccagggctacaggatgccccgtgtggagaactgcccagatgagctctatgacattatgaaaatgtgctggaaagaaaaggcagaagagagaccaacgtttgactacttacagagcgtcctggatgatttctacacagccacggaagggcaataccagcagcagccttaa

SEQ ID NO: 20 - protein, 759 aa, Artificial Sequence
  Synthetic Protein
MEQKLISEEDLGSGEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKGGSGGGSGGSNSTKVAVKTLKPGTMSVQAFLEEANLMKTLQHDKLVRLYAVVTREEPIYIITEYMAKGSLLDFLKSDEGGKVLLPKLIDFSAQIAEGMAYIERKNYIHRDLRAANVLVSESLMCKIADFGLARVIEDNEYTAREGAKFPIKWTAPEAINFGCFTIKSDVWSFGILLYEIVTYGKIPYPGRTNADVMTALSQGYRMPRVENCPDELYDIMKMCWKEKAEERPTFDYLQSVLDDFYTATEGQYQQQP

SEQ ID NO: 21 - DNA, 774 bp, Artificial Sequence
  Synthetic protein
  CDS (1)..(774)
atggtaggaagcctcaattgcatagtcgctgtgagtcaaaatatgggaattggcaaaaatggcgatctcccttggcccccactgagaaacgaatttaggtattttcaacggatgaccactacttctagcgtggaagggaaacagaacctcgtgataatgggtaaaaagacttggtttagcattccggaaaaaaatcggccacttaagggacgaataaatcttgtcttgtcaagggaattgaaggagccgccgcagggtgcccactttctcagtcggtcactggacgatgcgctgaagctgaccgaacagcccgagctcgccaacaaggtggatatggtgtggatagtcggcggtagttcagtctacaaagaagctatgaaccatccggggcatttgaaattgtttgttacgcgcattatgcaagattttgaaagtgatacctttttcccggagatagaccttgaaaaatataagttgcttcctgagtatccgggagtgctctcagatgtgcaagaagagaagggcggctctggcggcggctccggaggctctaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattctggttctggatacccatacgatgttccagattacgcttaa

SEQ ID NO: 22 - protein, 257 aa, Artificial Sequence
  Synthetic Protein
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGGSGGGSGGSTMTEKEIVDYVASQVTTAKKLRGGVVFVDEVPKGLTGKLDARKIREILIKAKKGGKIAVNSGSGYPYDVPDYA

SEQ ID NO: 23 - DNA, 1584 bp, Artificial Sequence
  Synthetic Protein
  CDS (1)..(1584)
atggagcagaagctgatcagcgaggaggacctgggttctggagaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaggcggctctggcggcggctccggaggctctggtattaagtacaagtttgaggtctatgaaaagaatgactaa

SEQ ID NO: 24 - protein, 527 aa, Artificial Sequence
  Synthetic Protein
MEQKLISEEDLGSGEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKGGSGGGSGGSGIKYKFEVYEKND

SEQ ID NO: 25 - DNA, 2190 bp, Artificial Sequence
  Synthetic protein
  CDS (1)..(2190)
atgctgatcagcctgctggaggtgatcgagcccgaggtgctgtacagcggctacgacagcaccctgcccgacaccagcaccaggctgatgagcaccctgaacaggctgggcggcaggcaggtggtgagcgccgtgaagtgggccaaggccctgcccggcttcaggaacctgcacctggacgaccagatgaccctgctgcagtacagctggatgagcctgatggccttcagcctgggctggaggagctacaagcagagcaacggcaacatgctgtgcttcgcccccgacctggtgatcaacgaggagaggatgcagctgccctacatgtacgaccagtgccagcagatgctgaagatcagcagcgagttcgtgaggctgcaggtgagctacgacgagtacctgtgcatgaaggtgctgctgctgctgagcaccgtgcccaaggacggcctgaagagccaggccgtgttcgacgagatcaggatgacctacatcaaggagctgggcaaggccatcgtgaagagggagggcggcggctctggcggcggctccggaggctctgatccaaaaaagaagagaaaggtagaggccagcggttcgggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaacactagtggcggctctggcggcggctccggaggctctagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggacccagcgcccgcagtgactcccgaggccagtcacctgttggaggaccccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttgacacatacaggtatatctaa

SEQ ID NO: 26 - protein, 729 aa, Artificial Sequence
  Synthetic Protein
MLISLLEVIEPEVLYSGYDSTLPDTSTRLMSTLNRLGGRQVVSAVKWAKALPGFRNLHLDDQMTLLQYSWMSLMAFSLGWRSYKQSNGNMLCFAPDLVINEERMQLPYMYDQCQQMLKISSEFVRLQVSYDEYLCMKVLLLLSTVPKDGLKSQAVFDEIRMTYIKELGKAIVKREGGGSGGGSGGSDPKKKRKVEASGSGRADALDDFDLDMLGSDALDDFDLDMLGSDALDDFDLDMLGSDALDDFDLDMLINTSGGSGGGSGGSSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFDTYRYI

SEQ ID NO: 27 - DNA, 4392 bp, Artificial Sequence
  Synthetic protein
  CDS (1)..(4392)
atgcaccaccaccaccaccacgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgatccaaaaaagaagagaaaggtaggcggctctggcggcggctccggaggctctaacagcagccagaactggcagaggttctaccagctgaccaagctgctggacagcatgcacgagatggtgggcggcctgctccagttctgcttctacaccttcgtgaacaagagcctgagcgtggagttccccgagatgctggccgagatcatcagcaaccagctgcccaagttcaaggccggcagcgtgaagcccctgctgttccaccagaagtaa

SEQ ID NO: 28 - protein, 1463 aa, Artificial Sequence
  Synthetic Protein
MHHHHHHDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDDPKKKRKVGGSGGGSGGSNSSQNWQRFYQLTKLLDSMHEMVGGLLQFCFYTFVNKSLSVEFPEMLAEIISNQLPKFKAGSVKPLLFHQK

SEQ ID NO: 29 - DNA, 2223 bp, Artificial Sequence
  Synthetic protein
  CDS (1)..(2223)
atggacgctctgtcacctgaacagctcgttttgacgcttttggaagctgaaccaccacacgttctgatcagccgaccctctgctccattcacagaggccagtatgatgatgagcctcactaagttggccgataaagaacttgtccatatgatttcctgggcgaagaaaatacccggattcgtagagctttccctcttcgatcaggtacggctgctcgagagttgctggatggaggttctgatgatggggctgatgtggagatccatagatcaccccggtaaacttatctttgcgccagaccttgtactcgaccgggacgaaggaaaatgcgtagaagggatcctggaaatttttgatatgctgctcgctaccaccagtcggttcagggagctgaaattgcagcacaaggaatatctttgcgtgaaagcgatgatacttctcaattcaagtatgtaccccctcgtgacagcaacccaggacgcagatagctccagaaaacttgctcatcttcttaatgcggtcaccgacgcattggtctgggttattgcaaagtctggtataggcggctctggcggcggctccggaggctctgatccaaaaaagaagagaaaggtagaggccagcggttcgggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaacactagtggcggctctggcggcggctccggaggctctagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggacccagcgcccgcagtgactcccgaggccagtcacctgttggaggaccccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttgacacatacaggtatatctaa

SEQ ID NO: 30 - protein, 740 aa, Artificial Sequence
  Synthetic Protein
MDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGIGGSGGGSGGSDPKKKRKVEASGSGRADALDDFDLDMLGSDALDDFDLDMLGSDALDDFDLDMLGSDALDDFDLDMLINTSGGSGGGSGGSSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFDTYRYI

SEQ ID NO: 31 - DNA, 4344 bp, Artificial Sequence
  Synthetic protein
  CDS (1)..(4344)
atgcaccaccaccaccaccacgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgatccaaaaaagaagagaaaggtaggcggctctggcggcggctccggaggctcttccagccagcaacaatctatgcgattggcgaacttgcttatgctgctcagtcacgtaaggcatgccagcaataaagggatggaacatcttcttaatatgaagtgtaagaatgtggtgcccgtttacgatctcctgctggaaatgctcaacgcacacgttctcaggtaa

SEQ ID NO: 32 - protein, 1447 aa, Artificial Sequence
  Synthetic Protein
MHHHHHHDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDDPKKKRKVGGSGGGSGGSSSQQQSMRLANLLMLLSHVRHASNKGMEHLLNMKCKNVVPVYDLLLEMLNAHVLR

SEQ ID NO: 33 - DNA, 2304 bp, Artificial Sequence
  Synthetic protein
  CDS (1)..(2304)
atgcacatggaggagctgcagaagagcatcggccacaagcccgagcccaccgacgaggagtgggagctgatcaagaccgtgaccgaggcccacgtggccaccaacgcccagggcagccactggaagcagaagaggaagttcctgcccgaggacatcggccaggcccccatcgtgaacgcccccgagggcggcaaggtggacctggaggccttcagccacttcaccaagatcatcacccccgccatcaccagggtggtggacttcgccaagaagctgcccatgttctgcgagctgccctgcgaggaccagatcatcctgctgaagggctgctgcatggagatcatgagcctgagggccgccgtgaggtacgaccccgagagcgagaccctgaccctgaacggcgagatggccgtgaccaggggccagctgaagaacggcggcctgggcgtggtgagcgacgccatcttcgacctgggcatgagcctgagcagcttcaacctggacgacaccgaggtggccctgctgcaggccgtgctgctgatgagcagcgacaggcccggcctggcctgcgtggagaggatcgagaagtaccaggacagcttcctgctggccttcgagcactacatcaactacaggaagcacggcggctctggcggcggctccggaggctctgatccaaaaaagaagagaaaggtagaggccagcggttcgggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaacactagtggcggctctggcggcggctccggaggctctagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggacccagcgcccgcagtgactcccgaggccagtcacctgttggaggaccccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttgacacatacaggtatatctaa

SEQ ID NO: 34 - protein, 767 aa, Artificial Sequence
  Synthetic Protein
MHMEELQKSIGHKPEPTDEEWELIKTVTEAHVATNAQGSHWKQKRKFLPEDIGQAPIVNAPEGGKVDLEAFSHFTKIITPAITRVVDFAKKLPMFCELPCEDQIILLKGCCMEIMSLRAAVRYDPESETLTLNGEMAVTRGQLKNGGLGVVSDAIFDLGMSLSSFNLDDTEVALLQAVLLMSSDRPGLACVERIEKYQDSFLLAFEHYINYRKHGGSGGGSGGSDPKKKRKVEASGSGRADALDDFDLDMLGSDALDDFDLDMLGSDALDDFDLDMLGSDALDDFDLDMLINTSGGSGGGSGGSSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFDTYRYI

SEQ ID NO: 35 - DNA, 4323 bp, Artificial Sequence
  Synthetic protein
  CDS (1)..(4323)
atgcaccaccaccaccaccacgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgatccaaaaaagaagagaaaggtaggcggctctggcggcggctccggaggctctcacgtgacccacttctggcccaagctgctgatgaaggtgaccgacctgaggatgatcggcgcctgccacgccagcaggttcctgcacatgaaggtggagtgccccaccgagctgttcccccccctgttcctggaggtgttcgagtaa

SEQ ID NO: 36 - protein, 1440 aa, Artificial Sequence
  Synthetic Protein
MHHHHHHDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDDPKKKRKVGGSGGGSGGSHVTHFWPKLLMKVTDLRMIGACHASRFLHMKVECPTELFPPLFLEVFE

SEQ ID NO: 37 - DNA, 2313 bp, Artificial Sequence
  Synthetic protein
  CDS (1)..(2313)
atgggcagccacatgttgaatcctgaaagcgctgatttgcgcgcacttgctaaacatctctacgattcttatatcaaatcattccccttgaccaaagcaaaagcgagagctatacttaccggtaagacaacggataagtccccattcgttatttatgacatgaattcacttatgatgggtgaagataagataaaattcaaacatataactcctctccaagagcaatcaaaggaagttgcgattagaatcttccagggctgtcaattcagaagcgtagaagcggtccaggagattacggagtatgcaaaatctatccccggctttgtcaacctcgaccttaacgaccaagtcactcttcttaaatacggggtacacgaaattatatacaccatgcttgcgtccctgatgaacaaagacggtgtgctgattagtgaaggtcaggggtttatgacgcgggaattccttaagagtcttagaaaacctttcggggactttatggagccaaagttcgagtttgcagtgaaatttaatgcactggaactcgacgactctgacctggctatattcatagccgttattattctcagcggagaccggcctggactcctcaacgtgaagccgattgaggacatacaggataacctgctccaagcccttgagctgcaactgaagctcaatcaccccgaatctagcggcggctctggcggcggctccggaggctctgatccaaaaaagaagagaaaggtagaggccagcggttcgggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaacactagtggcggctctggcggcggctccggaggctctagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggacccagcgcccgcagtgactcccgaggccagtcacctgttggaggaccccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtcc

SEQ ID NO: 38 - protein, 771 aa, Artificial Sequence
  Synthetic Protein
MGSHMLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSGGSGGGSGGSDPKKKRKVEASGSGRADALDDFDLDMLGSDALDDFDLDMLGSDALDDFDLDMLGSDALDDFDLDMLINTSGGSGGGSGGSSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLS

SEQ ID NO: 39 - DNA, 4287 bp, Artificial Sequence
  Synthetic protein
  CDS (1)..(4287)
atgcaccaccaccaccaccacgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgatccaaaaaagaagagacagctctttgccaagttgctccagaagatgactgacctgcgacaaatagtgacagagcacgtccaactgctccaagtgatcaaaaagaccgaaacggatatgagtcttcaccccctgttgcaggaaatatacaaggacctgtactaa

SEQ ID NO: 40 - protein, 1428 aa, Artificial Sequence
  Synthetic Protein
MHHHHHHDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDDPKKKRQLFAKLLQKMTDLRQIVTEHVQLLQVIKKTETDMSLHPLLQEIYKDLY

SEQ ID NO: 41 - DNA, 1752 bp, Artificial Sequence
  Synthetic protein
  CDS (1)..(1752)
atggcctgggagatcccccgggagtccatcaagttggtgaaaaggcttggcgctgggcagtttggggaagtctggatgggttactataacggcggctctggcggcggctccggaggctctgatccaaaaaagaagagaaaggtagaggccagcggttcgggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaacactagtggcggctctggcggcggctccggaggctctagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggacccagcgcccgcagtgactcccgaggccagtcacctgttggaggaccccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttgacacatacaggtatatctaa

SEQ ID NO: 42 - protein, 583 aa, Artificial Sequence
  Synthetic Protein
MAWEIPRESIKLVKRLGAGQFGEVWMGYYNGGSGGGSGGSDPKKKRKVEASGSGRADALDDFDLDMLGSDALDDFDLDMLGSDALDDFDLDMLGSDALDDFDLDMLINTSGGSGGGSGGSSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFDTYRYI

SEQ ID NO: 43 - DNA, 4914 bp, Artificial Sequence
  Synthetic protein
  CDS (1)..(4914)
atgcaccaccaccaccaccacgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgatccaaaaaagaagagaaaggtaggcggctctggcggcggctccggaggctctaacagtaccaaggtggctgtgaaaaccctgaagccaggaactatgtctgtgcaagccttcctggaagaagccaacctcatgaagaccctgcagcatgacaagctcgtgaggctctacgctgtggtcaccagggaggagcccatttacatcatcaccgagtacatggccaagggcagtttgctggatttcctgaagagcgatgaaggtggcaaagtgctgcttccaaagctcattgacttttctgctcagattgcagagggaatggcatacatcgagcggaagaactacattcaccgggacctgcgagcagctaatgttctggtctccgagtcactcatgtgcaaaattgcagattttggccttgctagagtaattgaagataatgagtacacagcaagggaaggtgctaagttccctattaagtggacggctccagaagcaatcaactttggatgtttcactattaagtctgatgtgtggtcctttggaatcctcctatacgaaattgtcacctatgggaaaattccctacccagggagaactaatgccgacgtgatgaccgccctgtcccagggctacaggatgccccgtgtggagaactgcccagatgagctctatgacattatgaaaatgtgctggaaagaaaaggcagaagagagaccaacgtttgactacttacagagcgtcctggatgatttctacacagccacggaagggcaataccagcagcagccttaa

SEQ ID NO: 44 - protein, 1637 aa, Artificial Sequence
  Synthetic Protein
MHHHHHHDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDDPKKKRKVGGSGGGSGGSNSTKVAVKTLKPGTMSVQAFLEEANLMKTLQHDKLVRLYAVVTREEPIYIITEYMAKGSLLDFLKSDEGGKVLLPKLIDFSAQIAEGMAYIERKNYIHRDLRAANVLVSESLMCKIADFGLARVIEDNEYTAREGAKFPIKWTAPEAINFGCFTIKSDVWSFGILLYEIVTYGKIPYPGRTNADVMTALSQGYRMPRVENCPDELYDIMKMCWKEKAEERPTFDYLQSVLDDFYTATEGQYQQQP

SEQ ID NO: 45 - DNA, 2184 bp, Artificial Sequence
  Synthetic protein
  CDS (1)..(2184)
atggtaggaagcctcaattgcatagtcgctgtgagtcaaaatatgggaattggcaaaaatggcgatctcccttggcccccactgagaaacgaatttaggtattttcaacggatgaccactacttctagcgtggaagggaaacagaacctcgtgataatgggtaaaaagacttggtttagcattccggaaaaaaatcggccacttaagggacgaataaatcttgtcttgtcaagggaattgaaggagccgccgcagggtgcccactttctcagtcggtcactggacgatgcgctgaagctgaccgaacagcccgagctcgccaacaaggtggatatggtgtggatagtcggcggtagttcagtctacaaagaagctatgaaccatccggggcatttgaaattgtttgttacgcgcattatgcaagattttgaaagtgatacctttttcccggagatagaccttgaaaaatataagttgcttcctgagtatccgggagtgctctcagatgtgcaagaagagaagggcggctctggcggcggctccggaggctctgatccaaaaaagaagagaaaggtagaggccagcggttcgggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaacactagtggcggctctggcggcggctccggaggctctagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggacccagcgcccgcagtgactcccgaggccagtcacctgttggaggaccccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttgacacatacaggtatatctaa

SEQ ID NO: 46 - protein, 727 aa, Artificial Sequence
  Synthetic Protein
MVGSLNCIVAVSQNMGIGKNGDLPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGGSGGGSGGSDPKKKRKVEASGSGRADALDDFDLDMLGSDALDDFDLDMLGSDALDDFDLDMLGSDALDDFDLDMLINTSGGSGGGSGGSSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFDTYRYI

SEQ ID NO: 47 - DNA, 4218 bp, Artificial Sequence
  Synthetic protein
  CDS (1)..(4218)
atgcaccaccaccaccaccacgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgatccaaaaaagaagagaaaggtaggcggctctggcggcggctccggaggctctggtattaagtacaagtttgaggtctatgaaaagaatgactaa

SEQ ID NO: 48 - protein, 1405 aa, Artificial Sequence
  Synthetic Protein
MHHHHHHDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDDPKKKRKVGGSGGGSGGSGIKYKFEVYEKND

SEQ ID NO: 49 - DNA, 21 bp, Artificial Sequence
  synthesized sequence
agatgtggaaacggaagaagc

SEQ ID NO: 50 - DNA, 673 bp, Artificial Sequence
  synthesized sequence
tttactgctgctcccgcttcttccgtttccacatcttgctggcagccggggtaccgcaggcaagtcgctttactgctgctcccgcttcttccgtttccacatctatggaatcacggcggccgcccgtatctcatcctttactgctgctcccgcttcttccgtttccacatctaatgcgtcggcgtgcggccgctgccagttgcatttactgctgctcccgcttcttccgtttccacatctttggctgtcctagggtacctggacgctccttgtttactgctgctcccgcttcttccgtttccacatcttggctggtgccgcaccggtaagcagaactagaggtggcaccggtttgattgcacagctttactgctgctcccgcttcttccgtttccacatcttgctggcagccggggtaccgcaggcaagtcgctttactgctgctcccgcttcttccgtttccacatctatggaatcacggcggccgcccgtatctcatcctttactgctgctcccgcttcttccgtttccacatctaatgcgtcggcgtgcggccgctgccagttgcatttactgctgctcccgcttcttccgtttccacatctttggctgtcctagggtacctggacgctccttgtttactgctgctcccgcttcttccgtttccacatct

(19) United States
(12) Patent Application Publication
(10) Pub. No.: US 2025/0321223 A1
(43) Pub. Date: Oct. 16, 2025

(54) ENGINEERING CHEMICALLY INDUCIBLE SPLIT PROTEIN ACTUATORS (CISPA)

(71) Applicant: KEMIJSKI INSTITUT, LJUBLJANA (SI)

(72) Inventors: Roman JERALA, Ljubljana (SI); Erik RIHTAR, Grosuplje (SI); Tina LEBAR, Brookline, MA (US)

(21) Appl. No.: 18/550,550

(22) PCT Filed: Mar. 15, 2022
(86) PCT No.: PCT/EP2022/056659

§ 371 Date: Sep. 14, 2023

(30) Foreign Application Priority Data
Mar. 15, 2021 (LU) LU102654

(51) Int. Cl. G01N 33/50; C07K 14/72; C12N 9/06; C12N 9/12; C12N 9/22; C12N 15/11; C12N 15/90

(52) U.S. Cl. CPC G01N 33/5023; C07K 14/721; C12N 9/003; C12N 9/12; C12N 9/226; C12N 15/11; C12N 15/907; C12Y 105/01003; C12Y 207/10002; G01N 33/5038; C07K 2319/61; C07K 2319/70; C07K 2319/80; C12N 2310/20; G01N 2500/10

(57) ABSTRACT

The present invention relates to chemically inducible split protein actuators (CISPA), which utilize ligand-binding proteins or protein domains originating from humans or other organisms, which are rationally split into two fragments that reassemble only in the presence of a cognate ligand. In particular, the invention relates to their design, manufacture, structure, and uses. The designed CISPAs can be used to regulate cellular processes such as gene expression, conditionally reconstitute of the function of a protein such as enzyme activity, as biological sensors, or for other applications.

### SEQUENCE LISTING

The sequence listing contained in the electronic file titled “008496184 sequence listing 20240805,” created 5 Aug. 2024 and comprising 422 kb is hereby incorporated herein.

### FIELD OF THE INVENTION

The present invention relates to chemically inducible split protein actuators (CISPA), and particularly, although not exclusively, to their design, manufacture, structure, and uses. The method of engineering CISPAs utilizes ligand-binding proteins or protein domains originating from humans or other organisms, which as described in this invention, are rationally split into two fragments that reassemble only in the presence of a cognate ligand, which is typically a small molecule. The designed CISPAs can be used to regulate cellular processes such as gene expression, conditionally reconstitute of the function of a protein such as enzyme activity, as biological sensors, or for other applications.

### BACKGROUND

Biological processes are often regulated by a complex network of protein-protein interactions. Therefore, the ability to precisely regulate protein interactions has a great potential for biological research and for therapeutic applications. The use of ligands as chemical input signals is desirable to trigger protein dimerization, as they are easy to use in vitro and in vivo (Stanton, Chory, & Crabtree, 2018). For this purpose chemically induced dimerization (CID), which employs ligand inducers to control homo- or hetero-dimerization of pairs of proteins, was developed as a powerful tool to regulate cellular processes in a tuneable and time-dependent manner.

One of the most widely used CID systems utilizes the immunosuppressive ligand rapamycin to induce heterodimerization of human derived proteins FKBP12 (FK506-binding protein) and FRB (FKBP-rapamycin-binding protein) (Derose, Miyamoto, & Inoue, 2013). Other examples of naturally occurring CID regulators include, abscisic acid-dependent AB11-PYL1 heterodimerization (Zhao et al., 2018) and gibberellin-dependent GID1-GA1 heterodimerization (Miyamoto et al., 2012). Each dimerization domain can be fused to a domain of effector proteins, the choice of which governs the downstream applications, ranging from sensing, control of protein localization, protein stability, signal transduction, protein secretion to controlling gene expression. For example, genetic fusion of heterodimerization domains to a DNA-binding domains (DBDs) and transcriptional activation domain (TAD) respectively, produces temporally regulated system where the addition of a ligand activates gene expression by recruiting the TAD into the proximity of the target gene promoter (Gao et al., 2016). Still other example includes modulation of enzyme activity, whereby ligand-induced dimerization mediates reconstitution of inactive split protein fragments (Fink et al., 2019). Furthermore, CID has also been used for gene therapy to induce the activation of therapeutically relevant molecules and responses (Pissios, Tzameli, Kushner, & Moore, 2000; Rivera et al., 1996; Ye et al., 1999). In one example, chemically induced dimerization was used for controlling the activity of chimeric antigen receptor (CAR)-based T cell therapies (Duong et al., 2019; Wu, Roybal, Puchner, Onuffer, & Lim, 2015). In one example CID was used to engineer the response to the thyroid hormone by separating the receptor protein in a way that none of the two segments interacts with the ligand (Pissios et al., 2000). In this case however the system exhibited high constitutive activity in the absence of a ligand. On the other hand, CID systems can be used as genetically encoded biosensors and offer a new mechanism for in vivo and in vitro small molecule detection. For example, CIDs can be applied for the point of care detection of small molecules such as drugs, hormones and toxins.

Despite the widespread use of CID tools, their clinical application has been limited due to the undesirable characteristics of the ligand or the non-human origin of protein components. For example, rapamycin is a potent immunosuppressant and as such less suitable for therapeutic application. Furthermore, a humanized chemically inducible system is needed to circumvent immune recognition and elimination of engineered cells (Schellekens, 2005). Additionally, there is a low diversity of ligands as regulators. For example, it would be very useful to have at our disposal several orthogonal systems that would allow simultaneous regulation of several different processes in human cells. There has been some recent success in expanding the repertoire of CIDs for new ligands, using methods such as in vitro selection of antibodies (Hill, Martinko, Nguyen, & Wells, 2018; Kang et al., 2019) and computational design (Foight et al., 2019; Glasgow et al., 2019), however these methods are time-consuming, expensive, labor-intensive, had low success rates and the designed proteins could trigger the response of the human immune system.

The present invention has been devised in light of the above considerations.

### SUMMARY OF THE INVENTION

In a first aspect, the invention relates to a method of designing a chemically inducible split protein actuator (CISPA), wherein the CISPA comprises two split fragments capable of forming a heterodimer in the presence of a ligand, the method comprising:
i. selecting a ligand-binding protein or protein domain, wherein the ligand-binding protein or protein domain is capable of binding a ligand,
ii. providing a 3D structure or molecular model of the ligand-binding protein or protein domain in complex with the ligand, and
iii. selecting a split site position within said 3D structure or model so as to divide the ligand-binding protein or protein domain into two split fragments.

In a second aspect, the invention relates to a method of producing a chemically inducible split protein actuator (CISPA), comprising the steps of designing a CISPA according to the first aspect, or providing a design for a CISPA produced according to the first aspect, and producing the split fragments according to the design.

In a third aspect, the invention relates to a chemically inducible split protein actuator (CISPA) comprising two split fragments capable of forming a heterodimeric ligand-binding protein or protein domain in the presence of a ligand.

In some embodiments, the two split fragments are unequal in size. In some embodiments, the smaller split fragment comprises no more than one third of the ligand-binding protein or protein domain, and the larger split fragment comprises the remainder of the ligand-binding protein or protein domain. In some embodiments, the majority of ligand interactions (i.e. between the CISPA heterodimer and its corresponding ligand) contact amino acid residues within the larger split fragment.

In some embodiments, the split fragments are fused to a first and a second segment of an effector protein or protein domain such that the function of the effector protein or protein domain is reconstituted when the heterodimer is formed in the presence of the ligand. In some embodiments, the effector protein or protein domain is a reporter which, when reconstituted, generates a detectable chemical or physical signal. In other embodiments, the effector protein or protein domain is a split protease, localization signal, DNA- or RNA-binding domain, recombinase, transcriptional regulator, or chromatin-remodelling domain, or a combination thereof. In some embodiments, the effector protein or protein domain is a transcriptional regulator.

In some embodiments, ligand-binding protein or protein domain is a human ligand-binding protein or protein domain. In some embodiments, the ligand-binding protein or protein domain is a nuclear receptor (NR) superfamily member, a Src family protein tyrosine kinase, dihydrofolate reductase (DHFR), or a fragment thereof. In some embodiments, the ligand-binding protein or protein domain is glucocorticoid receptor (GR), thyroid receptor beta (TRβ), peroxisome proliferator-activated receptor gamma (PPARγ) or estrogen receptor beta (ERβ), dihydrofolate reductase (DHFR), tyrosine protein kinase Lyn, tyrosine protein kinase Lck, tyrosine protein kinase Yes, tyrosine protein kinase Fyn, or a fragment thereof.

In some embodiments, the ligand is a human protein or fragment thereof, or a pharmacological compound. Preferably, the ligand has a molecular weight of 5 kDa or less.

In some embodiments, the CISPA comprises two split fragments which are unequal in size, and is selected from the following:
i. the smaller split fragment comprises amino acids 515-585 of SEQ ID NO:4 or a polypeptide having at least 80% identical amino acid residues thereof, the second split fragment comprises amino acids 1-179 of SEQ ID NO:2 or a polypeptide having at least 80% identical amino acid residues thereof, and the ligand is capable of binding a glucocorticoid receptor (GR);
ii. the smaller split fragment comprises amino acids 515-569 of SEQ ID NO:8 or a polypeptide having at least 80% identical amino acid residues thereof, the larger split fragment comprises amino acids 1-187 of SEQ ID NO:6 or a polypeptide having at least 80% identical amino acid residues thereof, and the ligand is capable of binding to ERP;
iii. the smaller split fragment comprises amino acids 515-562 of SEQ ID NO:12 or a polypeptide having at least 80% identical amino acid residues thereof, the larger split fragment comprises amino acids 1-214 of SEQ ID NO:10 or a polypeptide having at least 80% identical amino acid residues thereof, and the ligand is capable of binding to tRP;
iv. the smaller split fragment comprises amino acids 515-562 of SEQ ID NO:16 or a polypeptide having at least 80% identical amino acid residues thereof, the larger split fragment comprises amino acids 1-231 of SEQ ID NO:14 or a polypeptide having at least 80% identical amino acid residues thereof, and the ligand is capable of binding to PPARγ;
v. the smaller split fragment comprises amino acids 1-30 of SEQ ID NO:18 or a polypeptide having at least 80% identical amino acid residues thereof, the larger split fragment comprises amino acids 515-759 of SEQ ID NO:20 or a polypeptide having at least 80% identical amino acid residues thereof, and the ligand is capable of binding to Lyn; or
vi. the smaller split fragment comprises amino acids 515-527 of SEQ ID NO:24 or a polypeptide having at least 80% identical amino acid residues thereof, the larger split fragment comprises amino acids 1-174 of SEQ ID NO:22 or a polypeptide having at least 80% identical amino acid residues thereof, and the ligand is capable of binding to DHFR.

In a fourth aspect, the invention relates to a nucleic acid or set of nucleic acids encoding a CISPA according to the second or third aspects. In a fifth aspect, the invention relates to a vector or set of vectors encoding the nucleic acid or acids according to the fourth aspect. In a sixth aspect, a cell comprising the nucleic acid or acids of the fourth aspect, or the vector or vectors of the fifth aspect.

The invention also relates to applications and uses of the CISPAs of the invention.

In a seventh aspect, the invention relates to a method of detecting a ligand, comprising
i. contacting the ligand with a CISPA according to the second aspect, wherein the split fragments are fused to a first and a second segment of a reporter such that the function of the reporter is reconstituted when the heterodimer is formed in the presence of the ligand and so that, when reconstituted, the reporter generates a detectable chemical or physical signal, and
ii. measuring the detectable chemical or physical signal produced by the reporter.

In some embodiments, the ligand is a hormone. In some embodiments, the method of detecting a ligand is a method of detecting a hormone in a sample of bodily fluid or secretion.

In an eighth aspect, the invention provides a method of regulating transcription of a gene, comprising contacting a nucleic acid encoding the gene with a CISPA according to the first aspect, and contacting the CISPA with the ligand capable of binding the CISPA, wherein the split fragments are fused to a first and a second segment of a transcriptional regulator such that the function of the transcriptional regulator is reconstituted when the heterodimer is formed in the presence of the ligand.

In a ninth aspect, the invention provides a method of regulating a cellular process, comprising introducing a CISPA into a cell, and contacting the CISPA with the ligand capable of binding the CISPA, wherein the split fragments are fused to a first and a second segment of an effector protein or protein domain such that the function of the effector protein or protein is reconstituted when the heterodimer is formed in the presence of the ligand, and wherein the cellular process is regulated by the effector protein or protein domain.

In some embodiments of the seventh to ninth aspects, the method is performed in vitro. In other embodiments, the method is performed in vivo, and may optionally include the step of transforming a cell with nucleic acids or vectors encoding the CISPA, and/or expressing the CISPA from said nucleic acids or vectors within the cell.

The invention also relates to therapeutic applications of CISPAs according to the first aspect.

In a tenth aspect, the invention provides a method of treatment comprising
i. administering a therapeutic cell comprising or capable of expressing a CISPA to a patient in need thereof, and
ii. contacting the therapeutic cell with the CISPA ligand,
wherein the split fragments of the CISPA are fused to a first and a second segment of an effector protein or protein domain capable of regulating a therapeutic process such that the function of the effector protein or protein is reconstituted when the heterodimer is formed in the presence of the ligand.

In an eleventh aspect, the invention provides a method comprising
i. providing a cell derived from a patient in need of cell therapy, or from a donor, and
ii. modifying the cell to express a CISPA,
wherein the split fragments of the CISPA are fused to a first and a second segment of an effector protein or protein domain capable of regulating a therapeutic process such that the function of the effector protein or protein domain is reconstituted when the heterodimer is formed in the presence of the ligand.

In some embodiments, the therapeutic process is an immune response. In some embodiments, the effector protein or protein domain is a chimeric antigen receptor.

In some embodiments, the method further comprises the steps of
iii. administering the modified cell to the patient, and optionally
iv. contacting the therapeutic cell with the CISPA ligand.

The invention also provides a therapeutic cell for use in a method according to the tenth or eleventh aspect. The invention also provides the use of a therapeutic cell in the manufacture of a medicament for use according to the tenth or eleventh aspect.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1: Schematic presentation of engineering novel CISPAs and applications thereof. [A] Ligand binding protein or protein domain is split into two fragments (N- (nSplit) and C-terminal (cSplit) fragments) that reassemble only in the presence of a selected ligand. Thus, interaction between the split protein fragments is controlled by the presence and/or absence of the selected ligand. [B] Application of designed CISPAs for detection/sensing of selected ligands. Split fragments of CISPAs can be genetically fused to a split reporter protein. In the presence of a selected ligand, the split fragments of CISPA sensor reassemble, resulting in the measurable output signal by the reconstituted reporter. [C] Application of CISPAs for control of cellular processes, specifically control of gene expression. One split protein fragment of CISPAs is genetically fused to a DNA-binding domain (DBD), while the other split fragment is fused to a transcriptional activation (or repression) domain. When the cognate ligand is present, it causes the dimerization of split protein receptor fragments and recruitment of the transcriptional activation domain into the close proximity of a promoter, resulting in the transcription of gene of interest (goi). In an analogous way gene expression may be repressed by genetically fusing a repression domain to the split receptor domain.

FIG. 2: Shows the embodiment of ligand mediated CISPA sensing based on split ligand binding domain (LBDs) of nuclear receptor superfamily (NRs) members. [A] Schematic representation of CISPA sensor based on split ligand binding domains (LBDs) of nuclear receptor superfamily (NRs) members. Split fragments (nSplit and cSplit) of ligand binding domains (LBDs) are fused to a split firefly luciferase reporter (nLuc and cLuc). When a specific cognate ligand is present, the split fragments of CISPA sensor will reassemble. Following this dimerization, the split reporter fragments (nLuc and cLuc) will come together, leading to their reconstitution and measurable output signal (emitted light). Dose response curves for CISPA sensors based on split LBDs of glucocorticoid receptor (GR2) [B], estrogen receptor beta (ERβ) [C], thyroid receptor beta (TRβ) [D], peroxisome proliferator-activated receptor gamma (PPARγ) [E]. 50 ng of each plasmid encoding CISPA sensor pair was transiently co-transfected in HEK293T cells which were 24 h post-transfection stimulated with the indicated ligands. Concentration dependent increase in luciferase activity indicates the dimerization of split protein fragments of CISPA sensor. Replicates represent HEK293T cell cultures, individually transfected with the same mixture of plasmids. The values represent the mean and standard deviation of replicates within two to three independent experiments (n=4). Data was plotted as a standard dose response curve. “COR”, “DEX”, “MOF”, “EST”, “GEN”, “OHT”, “SOB”, “T3” and “ROS” represent the cortisol, dexamethasone, mometasone furoate, 17β-estradiol, genistein, 4-hydroxytamoxifen, triiodothyronine, sobetirome and rosiglitazone, respectively.

FIG. 3: Shows the embodiment of ligand mediated CISPA sensing based on split kinase domain of tyrosine protein kinase Lyn [A] or dihydrofolate reductase (DHFR) [B]. 50 ng of each plasmid encoding CISPA sensor pair was transiently co-transfected in HEK293T cells. After 24 h HEK293T cells were stimulated with the increasing concentration of indicated ligands. Concentration dependent increase in luciferase activity indicates the dimerization of split protein fragments of CISPA sensor. Replicates represent HEK293T cell cultures, individually transfected with the same mixture of plasmids. The values represent the mean and standard deviation of replicates within two to three independent experiments (n=4). Data was plotted as a standard dose response curve. “DAS”, “MTX” and “PTX” represent dasatinib, methotrexate and pralatrexate, respectively.

FIG. 4: Shows the embodiment of CISPA systems for ligand-mediated control of transcriptional activation. [A] Schematic representation of ligand-mediated activation of reporter gene firefly luciferase (fLuc). The nSplit fragments of CISPAs are fused to transcriptional activation domain VPR and cSplit fragments to S. pyogenes catalytically inactive Cas9 (dCas9) DNA-binding domain. When the cognate ligand is present, it causes the dimerization of split protein fragments and recruitment of the transcriptional activation domain VPR into the close proximity of a minimal promoter (pMin), driving the expression of a firefly luciferase reporter gene. [B] Ligand-dependent activation of the reporter gene in HEK293T cells employing CISPAs based on split ligand binding domains (LBDs) of glucocorticoid receptor (GR2), estrogen receptor beta (ERβ), thyroid receptor beta (TRβ), peroxisome proliferator-activated receptor gamma (PPARγ), kinase domain of tyrosine protein kinase Lyn and dihydrofolate reductase (DHFR). HEK293T cells were co-transfected with 50 ng of the reporter plasmid (10[AB]_Pmin_fLuc), 25 ng of gRNA ([AB]nt) encoding plasmid along with 25 ng of dCas9:cSplit and 50 ng of nSplit:VPR fusion encoding plasmids. Replicates represent HEK293T cell cultures, individually transfected with the same mixture of plasmids. The values represent the mean and standard deviation of replicates within two independent experiments (n=4). Fold activation was calculated by normalizing the RLU values of each sample to the RLU value of the non-stimulated reporter only (mock) control within the same experiment.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Definitions

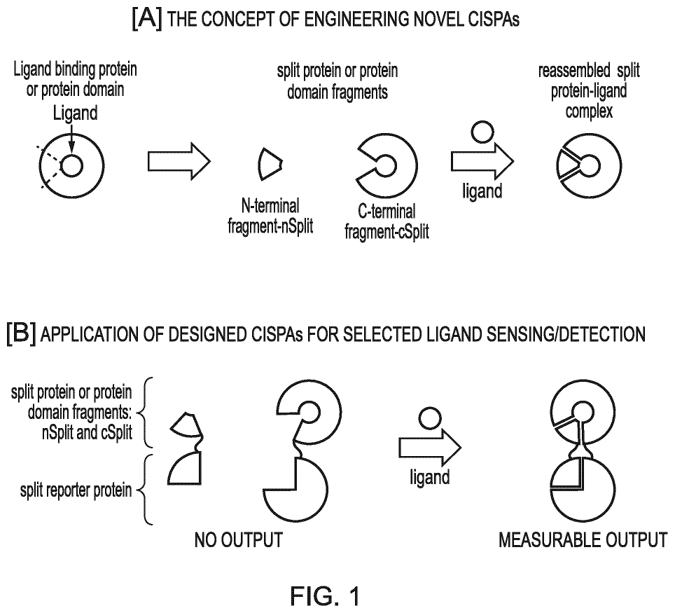

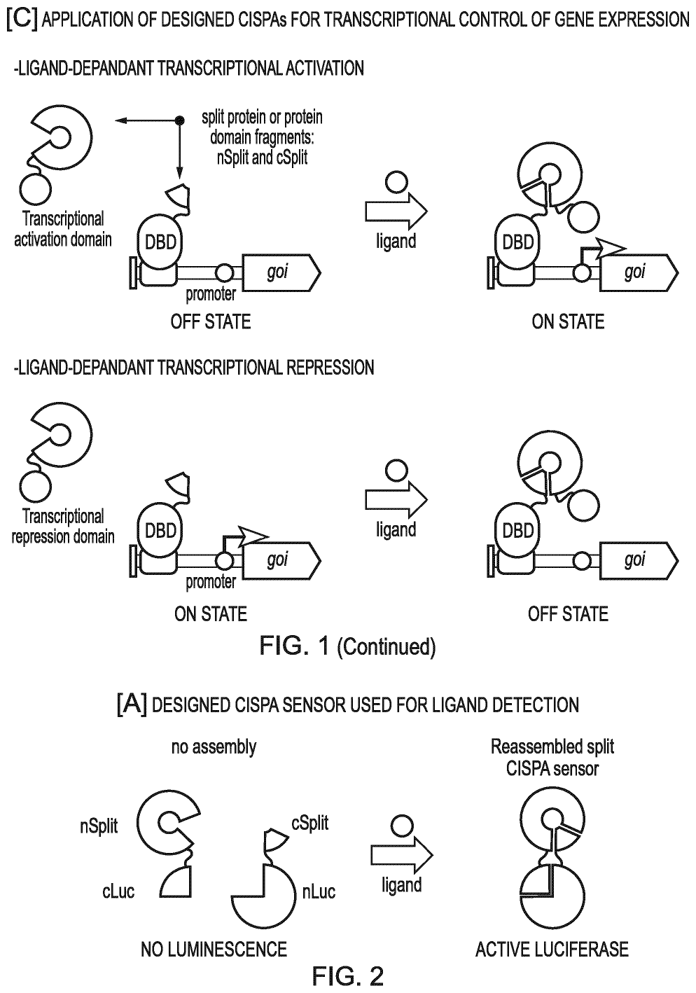

The present disclosure refers to the design of chemically inducible split protein actuators (CISPA). The disclosed CISPAs are based on preferably human derived ligand binding proteins or ligand binding protein domains, which are divided into two fragments (N- and C-fragment) that reassemble in the presence of a cognate ligand (FIG. 1A). Thus, interaction between the split protein fragments is controlled by the presence and/or absence of the selected ligand. This strategy of CISPA design is inspired by rationally designed split proteins, but unlike previous split proteins we use ligands that originally bind these intact proteins to induce reassembly of split protein fragments. The presented invention also includes applications of the CISPAs.

In the particular embodiment, the CISPA refers to split proteins or split protein domains originating from humans or other organisms, preferably with known tertiary structure ligand-protein complex, selected from protein 3D structure databases (e. g. PDB) or a reliable 3D model (obtained e.g. from Swiss Model database) that help in the design of split site. The N-terminal fragment of the selected split protein or protein domain is referred to as nSplit, while the C-terminal fragment is referred to as cSplit. The invention specifies that the split site positions are preferably selected within the less structured solvent-exposed loops. Additionally, the split site positions are preferably selected so that one of the fragments (nSplit or cSplit) is substantially smaller than the other, the smaller fragment comprising one to three segments of protein secondary structure such as helices or beta strands and the larger fragment comprises more amino acid residues than the smaller fragment. Additionally the larger fragment may comprise the majority of the contacts (preferably at least 70%) between the protein and the cognate ligand. Both nSplit and cSplit fragments reassemble only in the presence of a selected ligand.

In some embodiments, the smaller fragment may comprises the majority of the contacts (preferably at least 70%) between the protein and the cognate ligand.

The disclosed CISPAs are preferably based but not limited to human derived ligand binding proteins or protein domains, which are divided into two or more split protein or protein domain fragments (nSplit and cSplit) that reassemble in the presence of a selected ligand. Each of two split fragments may be genetically fused to protein domains that when brought in proximity result in new structure or function, such as catalytic activity, transcriptional activation or others.

The term “split protein or protein domain fragments”, as used herein, refers to two or more polypeptides, each of them being equal to one part of the whole protein or protein domain. In the absence of the selected ligand the split fragments do not reassemble. The split protein or protein domain fragments reassemble only in the presence of a cognate ligand. Thus, interaction between the split protein or split protein domain fragments is controlled by the presence and/or absence of the cognate ligand.

The terms “nSplit” and “cSplit” refer respectively to the CISPA split protein or protein domain fragments which contain and correspond to the N-terminal and C-terminal regions of the ligand-binding protein or protein domain.

The term “protein”, as used herein, refers to the polymeric form of amino acids of any length, which expresses any function, for instance localizing to a specific location, localizing to specific DNA sequence, facilitating and triggering chemical reactions, transcription regulation, structural function, and biological recognition.

The term “protein domain”, used herein, refers to a folding functional unit of a protein. For example a part of a protein that can fold and be expressed independently of the whole protein and is typically composed of one or more secondary structure elements, such as alpha helices or beta strands.

The term “ligand binding domain (LBD)” as used herein refers to a highly structurally conserved domain within a protein that is responsible for ligand (e. g. endogenous hormones, vitamins A and D, fatty acids and other) binding. An LBD may typically contain 11-13 alpha-helices.

### Spill Site—3Ary Structure

The split site position between the two fragments is preferably selected within the less structured solvent-exposed loops of a selected protein with known tertiary structure of ligand-protein complex or a molecular model of the complex, using established methods of molecular modelling and docking.

### Smaller/Larger Fragment

Additionally, the split site positions are preferably selected so that one of the two fragments is smaller than the other.

For example, the smaller fragment (nSplit or cSplit) may comprise one, two, or three segments of protein secondary structure such as e.g. alpha helices or beta strands, while the larger fragment (nSplit or cSplit) comprises larger number of amino acid residues that the smaller fragment. In some embodiments, the smaller fragment comprises at least one segments of protein secondary structure. In some embodiments, the smaller fragment comprises no more than three segments of protein secondary structure Additionally the larger fragment forms the majority of contacts between the protein and the cognate ligand. Thus, interaction between the split protein fragments is controlled by the presence and/or absence of the selected cognate ligand. The important advantage of the disclosed CISPAs is the use of human derived proteins or protein domains, as they do not activate immune response against cells expressing CISPAs, as is true in the case of chemically inducible dimerization systems originating from proteins encoded by another organism or that have been designed. Still another advantage is the engineering principle disclosed here to design CISPAs, which is universal and could be used to create novel CISPAs based on almost any ligand binding protein or protein domain.

In some embodiments, the larger fragment forms more than 50% of the contacts between the ligand-binding protein or protein domain and the cognate ligand. In some embodiments, the larger fragment forms at least 60%, at least 70%, at least 75%, at least 80%, at least 85% or at least 90% of the contacts between the ligand-binding protein or protein domain and the cognate ligand. In some embodiments, the larger fragment forms more than 70% and less than 100%, more than 70% and less than 95%, or more than 70% and less than 90% of the contacts between the ligand-binding protein or protein domain and the cognate ligand. In some embodiments, the larger fragment does not form 100% of contacts between the ligand-binding protein or protein domain and the cognate ligand, and the smaller fragment forms at least one contact between the ligand-binding protein or protein domain and the cognate ligand

### Ligands

The term “ligand”, used herein, refers to any small molecule with low molecular weight (less than or equal to 5000 Daltons, preferably less than or equal to 4000 Daltons, preferably less than or equal to 3000 Daltons, preferably less than or equal to 2000 Daltons, preferably less than or equal to 1000 Daltons, preferably less than or equal to 900 Daltons, preferably less than or equal to 800 Daltons, preferably less than or equal to 700 Daltons, preferably less than or equal to 600 Daltons, more preferably less than or equal to 500 Daltons). The said ligands include but are not limited to for example lipids, monosaccharide, second messengers, hormones, inhibitors, other natural products and metabolites, as well as drugs and other synthetic small molecules.

### Preferred CISPA Proteins

Exemplary CISPAs include those based on split ligand binding domains (LBDs) of nuclear receptor superfamily (NRs) members, for example, but not limited to LBDs of glucocorticoid receptor (GR), thyroid receptor beta (TRβ), peroxisome proliferator-activated receptor gamma (PPARγ) and estrogen receptor beta (ERβ). The present invention also refers to CISPAs based on split human dihydrofolate reductase (DHFR). The present invention also refers to CISPAs based on split kinase domain of Src kinase family members (for example, tyrosine protein kinase Lyn, tyrosine protein kinase Lck, tyrosine protein kinase Yes, and/or tyrosine protein kinase Fyn).

The method of engineering is exemplified by the CISPAs based on split protein or protein domain fragments, including:
(i) Split ligand-binding domains (LBDs) of nuclear receptors (NRs) superfamily members. For example, the split LBD domain of the glucocorticoid receptor (GR) that binds cortisol or synthetic glucocorticoids such as dexamethasone, mometasone furoate and others. Another example of CISPAs with split-LBD is the thyroid receptor beta (TRβ) that binds the natural hormone triiodothyronine and synthetic derivatives such as sobetirome (GC-1). Another example is based on split LBD of peroxisome proliferator-activated receptor gamma (PPARγ) and estrogen receptor beta (ERβ), which bind various natural and synthetic ligands. Specifically, LBD domain of PPARγ binds various natural polyunsaturated fatty acids such as arachidonic acid and arachidonic acid metabolites as well as synthetic small molecules, exemplified by a class of small molecules belonging to the group of thiazolidinediones, and other related small molecules. In another example LBD domain of ERB binds a variety of natural estrogens (17β-estradiol, genestein-phytoestrogen and others) as well as other synthetic estrogens (ethinylestradiol) and non-steroidal ligands (tamoxifen, raloxifene).
(ii) Split human dihydrofolate reductase (DHFR) that binds small molecule inhibitors such as methotrexate, pralatrexate and others.
(iii) Split kinase domain of tyrosine protein kinase Lyn that binds a variety of inhibitors such as dasatinib, imatinib and others.

The chosen implementation examples are used merely to best describe the invention and its applicability, and have no intention on limiting the scope of the invention, as many other human derived ligand binding proteins or protein domains may be used to design CISPAs according to the said description of the invention.

The term “nuclear receptors” (NRs) as used herein refers to a superfamily of proteins with a modular domain organization: a DNA-binding domain (DBD) and a ligand-binding domain that are linked via a hinge region. The nuclear receptor superfamily includes receptors for the glucocorticoids (GR), mineralocorticoids (MR), estrogens (ER), progestins (PR), and androgens (AR), as well as receptors for peroxisome proliferators (PPARs), vitamin D (VDR), and thyroid hormones (TR). Nuclear receptors regulate expression of specific genes, depending on the presence of their cognate ligands that control the development, homeostasis, metabolism and other cellular processes.

An exemplary CISPA is based on the glucocorticoid receptor (GR2). The nSplit and cSplit polypeptide fragments are selected from the split ligand binding domain of glucocorticoid receptor (GR2); nSplit comprises amino acids 1-179 of SEQ ID NO: 2 or a polypeptide having at least 80%, at least 90%, at least 95% or 100% identical amino acid residues thereof; and cSplit comprises amino acids 515-585 of SEQ ID NO: 4 or a polypeptide having at least 80%, at least 90%, at least 95% or 100% identical amino acid residues thereof.

Another exemplary CISPA is based on the split ligand binding domain of estrogen receptor beta (ERβ). The nSplit and cSplit polypeptide fragments are selected from the split ligand binding domain of estrogen receptor beta (ERβ); nSplit comprises amino acids 1-187 of SEQ ID NO: 6 or a polypeptide having at least 80%, at least 90%, at least 95% or 100% identical amino acid residues thereof; and cSplit comprises amino acids 515-569 of SEQ ID NO: 8 or a polypeptide that having at least 80%, at least 90%, at least 95% or 100% identical amino acid residues thereof.

In another exemplary CISPA, the nSplit and cSplit polypeptide fragments are selected from the split ligand binding domain of thyroid receptor beta (TRβ); nSplit comprises amino acids 1-214 of SEQ ID NO: 10 or a polypeptide having at least 80%, at least 90%, at least 95% or 100% identical amino acid residues thereof; and cSplit comprises amino acids 515-562 of SEQ ID NO: 12 or a polypeptide having at least 80%, at least 90%, at least 95% or 100% identical amino acid residues thereof.

In another exemplary CISPA, the nSplit and cSplit polypeptide fragments are selected from the split ligand binding domain of peroxisome proliferator-activated receptor gamma (PPARγ); nSplit comprises amino acids 1-231 of SEQ ID NO: 14 or a polypeptide having at least 80%, at least 90%, at least 95% or 100% identical amino acid residues thereof; and cSplit comprises amino acids 515-562 of SEQ ID NO: 16 or a polypeptide having at least 80%, at least 90%, at least 95% or 100% identical amino acid residues thereof.

In another exemplary CISPA, the nSplit and cSplit polypeptide fragments are selected from the kinase domain of tyrosine protein kinase Lyn; nSplit comprises amino acids 1-30 of SEQ ID NO: 18 or a polypeptide having at least 80%, at least 90%, at least 95% or 100% identical amino acid residues thereof; and cSplit comprises amino acids 515-759 of SEQ ID NO: 20 or a polypeptide having at least 80%, at least 90%, at least 95% or 100% identical amino acid residues thereof.

In another exemplary CISPA, the nSplit and cSplit polypeptide fragments are selected from the human dihydrofolate reductase (DHFR); nSplit comprises amino acids 1-174 of SEQ ID NO: 22 or a polypeptide having at least 80%, at least 90%, at least 95% or 100% identical amino acid residues thereof; and cSplit comprises amino acids 515-527 of SEQ ID NO: 24 or a polypeptide having at least 80%, at least 90%, at least 95% or 100% identical amino acid residues thereof.

### Effector Domains

In some embodiments, the CISPA split protein fragments are fused to segments of an effector protein or protein domain. When the CISPA heterodimer is formed following binding to the ligand, the segments are brought into close proximity and the effector protein or protein domain is reconstituted. Each CISPA split protein fragment is fused to a different segment of the effector protein, such that when the CISPA heterodimer is formed, the entire effector protein or protein domain is reconstituted. Each segment of the effector protein or protein domain is unable to perform the function of the effector protein alone, but the reconstituted effector protein or protein domain is able to perform this function. An effector protein or domain may be a naturally occurring protein or domain thereof, or may be an engineered domain.

In some embodiments, the CISPA split protein fragments are fused to segments of a first effector protein or protein domain and segments of a further effector protein or protein domain, such that ligand binding results in the reconstitution of the first and further effector protein or protein domains. The first and further effector proteins or protein domains may be identical.

The term “effector protein” or “effector protein domain”, in the description refers to any protein domain with a specific function, for example, but not limited to nuclease domains, recombinases, catalytic, transcriptional activation domains and chromatin silencing domains.

The CISPA split protein fragments and segments of an effector protein or protein domain may be fused via a genetic fusion. The term “genetic fusion”, used herein, refers to the polypeptide or nucleic acid that encodes for the polypeptide in a single chain that comprises polypeptide of two or more constituents that are consecutive or between them are short linker polypeptides that prevent steric overlap, typically comprising 1-10 small polar flexible amino acid residues, typically glycine or serine or similar amino acid residues.

In some embodiments, the effector protein or protein domain is a reporter which, when reconstituted generate a chemical or physical signal that can be detected by chemical, physical or biological methods. A CISPA fused to a reporter therefore reports the concentration of the target ligand in vitro or in vivo. Exemplary reporters include split luciferase, split fluorescent protein, split glucose oxidase or other split proteins.

In some embodiments, the effector protein or protein domain controls or regulates (i.e. up or down-regulates) a cellular process. Exemplary cellular processes include gene expression, protein localization, protein stabilization, signal transduction, reconstitution of the function of a protein (such as enzymatic activity of split proteases, kinases, phosphatases and others) or any other cellular processes, wherein each of the split ligand binding protein fragments of CISPAs is fused to a domain of the effector protein. Examples of effector proteins are selected among the split proteases, localization signals, DNA- or RNA-binding domains, recombinases, transcriptional activators/repressors, chromatin-remodelling domains, or any other proteins involved in biologically relevant processes, or combinations thereof. In these embodiments, the ligand acts as an external switch to activate or inhibit the cellular process.

In some embodiments, the effector protein or protein domain controls or regulates (i.e. up or down-regulates) a therapeutic process. Exemplary therapeutic processes include T-cell cancer immunotherapy based on chimeric antigen receptors or stem cell regeneration of differentiation based on artificial cells. Examples of effector proteins capable of controlling a therapeutic response include chimeric antigen receptors (CAR). In these embodiments, the ligand acts as an external switch to activate or inhibit the therapeutic process.

### Cells, Nucleic Acids, Vectors

Nucleic acids encoding a CISPA as described herein, for example a CISPA fused to an effector protein or protein domain. A nucleic acid may encode a single CISPA split protein, along with any effector protein segments fused to it. Alternatively, a single nucleic acid may encode an entire CISPA.

The term “nucleic acid”, used herein, refers to a polymeric form of nucleotides (ribonucleotides or deoxyribonucleotides) of any length and is not limited to single, double or higher number of chains of DNA or RNA, genomic DNA, cDNA, DNA-RNA hybrids, or polymers with a phosphorothioate polymer backbone made from purine and pyrimidine bases or other natural, chemical or biochemically modified, synthetic or derived nucleotide bases.

The term “recombinant”, used herein, means that a particular nucleic acid (DNA or RNA) is a product of various combinations of cloning, restriction and/or ligation or chemical synthesis leading to a construct having structurally coding or non-coding sequences different from endogenous nucleic acids in a natural host system.

A nucleic acid may be operably linked to a regulatory sequence. In this specification the term “operably linked” may include the situation where a selected nucleotide sequence and regulatory nucleotide sequence are covalently linked in such a way as to place the expression of a nucleotide sequence under the influence or control of the regulatory sequence. Thus a regulatory sequence is operably linked to a selected nucleotide sequence if the regulatory sequence is capable of effecting transcription of a nucleotide sequence which forms part or all of the selected nucleotide sequence. Where appropriate, the resulting transcript may then be translated into a desired protein or polypeptide.

The nucleic acid may be comprised within a vector. Exemplary vectors include transformation vectors such as viral vectors (such as retroviral and adenoviral vectors), artificial chromosomes (yeast artificial chromosomes, yeast artificial chromosomes), bacterial expression vectors, and the like. A vector may additionally include a regulatory sequence, a selection marker (for example, a resistance marker), an origin of replication (ORF) or an additional coding nucleotide sequence encoding a gene, such as a gene for facilitating the transformation of and/or retention of the vector within a host cell.

The disclosure also describes cells comprising the vectors or nucleic acids encoding a CISPA as described herein. The term “cell”, used herein, refers to a eukaryotic or prokaryotic cell, a cellular or multicellular organism (cell line) cultured as a single cell entity that has been used as a recipient of nucleic acids and includes the daughter cells of the original cell that has been genetically modified by the inclusion of nucleic acids. The term refers primarily to cells of higher developed eukaryotic organisms, preferably vertebrates, preferably mammals. This invention relies also on non-vertebrates cells, preferably plant cells.

The term “cells” also refers to human or animal primary cells or cell lines. Naturally, the descendants of one cell are not necessarily completely identical to the parents in morphological form and its DNA complement, due to the consequences of natural, random or planned mutations.

Cells may be genetically modified so as to comprise a vector or nucleic acids encoding a CISPA. A “genetically modified host cell” (also “recombinant host cell”) is a host cell into which the nucleic acid has been introduced. The eukaryotic genetically modified host cell is formed in such a way that a suitable nucleic acid or recombinant nucleic acid is introduced into the appropriate eukaryotic host cell. The invention hereafter includes host cells and organisms that contain a nucleic acid according to the invention (transient or stable) bearing the operon record according to the invention. Suitable host cells are known in the field and include eukaryotic cells. It is known that proteins can be expressed in cells of the following organisms: human, rodent, cattle, pork, poultry, rabbits and the like. Host cells may include cultured cell lines of primary or immortalized cell lines.

The insertion of the vectors into the host cells is carried out by conventional methods known from the field of science, and the methods relate to transformation or transfection and include e.g.: chemically induced insertion, electroporation, micro-injection, DNA lipofection, cellular sonication, gene bombardment, viral DNA input, as well as other methods. The entry of DNA may be of transient or stable. Transient refers to the insertion of a DNA with a vector that does not incorporate the DNA of the invention into the cell genome. A stable insertion is achieved by incorporating DNA of the invention into the host genome. The insertion of the DNA of the invention, in particular for the preparation of a host organism having stably incorporated a nucleic acid, e.g. a DNA, of the invention, can be screened by the presence of markers. The DNA sequence for markers refers to resistance to antibiotics or chemicals and may be included on a DNA vector of the invention or on a separate vector.

This invention includes a CISPA, wherein the split proteins forming CISPA are produced in living cells or by in vitro methods, such as chemical synthesis or in vitro transcription and translation.

### Sensing

The present invention refers to the use of CISPAs can be used as a sensor for in vitro or in vivo sensing of selected ligands and respond directly using different reporter output signals or function or change of property. For example, split protein fragment (nSplit and cSplit) can be genetically fused to a split reporter protein. In the presence of selected ligand, the split fragments of CISPA sensor will reassemble. Following this dimerization of the CISPA sensor domain, the fused reporter fragments will also come together, leading to their reconstitution and measurable output signal. Also disclosed is a sensor for detecting a ligand, comprising a CISPA fused to a split reporter protein.

In one embodiment, the CISPAs can be used for in vitro or in vivo sensing of selected ligands and to respond directly to their presence with various reporter output signals. For example, as exemplified in FIG. 1B, both nSplit and cSplit protein fragments can be genetically fused to fragments of a split reporter protein. In the presence of a selected ligand, the split fragments of a CISPA sensor reassemble. Following the nSplit and cSplit assembly, the reporter fragments will also come together, leading to their reconstitution and a measurable output signal. Examples of split reporters are selected among the split luciferases, fluorescent proteins, phosphatases, proteases, oxidoreductases and other proteins known in the field that can generate a measurable output signal but require proximity to reconstitute their function.

The term “sensor”, used herein, refers to a molecule or molecular complex where the presence of a cognate ligand triggers the generation of measurable output signal as e.g. emitted light, fluorescence, electric current, or other chemical or physical signal or change of physicochemical property that correlates with the addition of a cognate ligand.

In some embodiments, the sensor is for detecting a ligand such as a hormone in body fluids, serums, or secretions, and comprises a CISPA capable of binding said hormone. Methods of detecting such a ligand may comprise obtaining or providing a sample of the bodily fluid, serum or secretion.

### Gene Expression

In some embodiments, the CISPAs can be used for in vitro or in vivo regulation of gene expression. For example, split protein fragments (nSplit and cSplit) can be genetically fused to a split transcriptional regulator. As used herein, a “transcriptional regulator” is a protein or protein domain capable of regulating (upregulating or downregulating) transcription of a target gene. For example, as exemplified in FIG. 1C, one split protein fragment of the CISPAs can be genetically fused to a DNA-binding domain and the other split fragment can be fused to a transcription activation domain. When the selected ligand is present, it causes the dimerization of the nSplit and cSplit protein fragments, resulting in the recruitment of the transcription activation domain into the close proximity of a promoter, causing transcription of the target gene. Therefore, CISPAs based on split proteins provide a powerful tool for controlling gene expression in many cells, including but not limited to human cells.

Any “target gene” may be used. Use of a transcriptional regulator CISPA fusion provides a means of exerting external control over the transcription of a target gene, effectively providing an external regulatory “switch”, and may find many applications in synthetic biology, genetic engineering, cell culture, and therapeutic fields, simply by changing the target gene and/or cell within which transcription is regulated. The CISPA may be transcribed and/or translated within a cell containing the target gene, may be introduced to the cell as a polypeptide, or the target gene may be contacted with the CISPA outside the cellular context.

An exemplary DNA binding domain is a catalytically inactive Cas9 (dCas9) DNA binding domain, which may be paired with any transcriptional regulatory domain, so long as the transcriptional regulatory domain is incapable of DNA binding. Thus, regulation of gene expression only occurs when the CISPA binds the ligand and brings dCas9 and the transcriptional regulatory domain into proximity. Advantageously, the binding specificity of dCas9 is determined through the use of a guide RNA. Thus, a gene expression regulating CISPA utilising a dCas9 DNA binding domain may be used to regulate the expression of any gene simply by changing the guide DNA. In methods using such a CISPA, the CISPA may be contacted with the ligand in the presence of the guide protein. The guide DNA may be administered with the CISPA, with the ligand, or may be transcribed in vivo or in vitro. These techniques are known as CRISPR activation (aCRISPR) and CRISPR interference (CRISPRi), depending on whether the transcriptional regulatory domain is a positive or negative regulator.

Transcriptional regulators include transcriptional activators, comprising one or more transcription factors in fusion, for example one or more basal transcription factors. A suitable transcriptional activator for use with dCas9 is the activator VP64-p65-Rta (VPR), comprising a fusion between the VP64, p65 and Rta transcription factors. Whilst Vp64 may be used alone, the use of three transcription factors results in increased expression of targeted gene. Another regulator is synergistic activation mediator (SAM), comprising a fusion between MS2, p65, and HSF1 proteins, recruits various transcriptional factors working synergistically to activate the gene of interest. Alternatively, a Suntag domain, as described in Tanenbaum et al, 2014, may be used. The Suntag domain comprises a series of antibody epitopes, which are capable of binding transcription factor-antibody fusions. Suitable transcriptional suppressor domains include the Kruppel associated box (KRAB) domain. In some embodiments the transcriptional suppressor is a split-dCas9, where formation of the CISPA heterodimer results in the reconstitution of dCas9, which blocks transcription through steric hindrance.

In some embodiments, nSplit and cSplit are provided to a cell, such as by being expressed from the corresponding nucleic acids encoding the respective protein fragments introduced into the cells. In the absence of the cognate ligand inducers, nSplit and cSplit do not reassemble and there is no effect on cellular processes. When the selected cognate ligand is present, it causes the dimerization of nSplit and cSplit protein fragments, and any other effector protein that has been genetically fused to them. Examples of effector proteins are selected among the proteases, localization signals, DNA or RNA binding domains, recombinases, transcriptional activators/repressors, chromatin-remodelling domains, or any other proteins involved in biologically relevant processes.

The term “DNA-binding domain”, used herein, refers to any protein domain with the ability to bind a DNA molecule. The DNA-binding protein could be of natural origin or artificially designed whole protein or only a segment with characteristic to bind to nucleic acid in sequence specific manner. Exemplary DNA-binding domains include DNA-binding zinc finger, TALE, Cas/gRNA combination, and helix-turn-helix transcription factor domains.

### Therapy

CISPAs, and particular CISPA-effector protein fusions, as described herein may find use in methods of therapy. As used herein, “therapy” means any method of preventing, treating, or ameliorating a symptom of a disease or pathological condition, temporarily or permanently.

This disclosure includes a CISPA for control of cell therapy by adding a CISPA ligand as an external signal to activate or inhibit a therapeutic processes under the regulation of the effector protein or protein domain. For example, the therapeutic process may be T-cell cancer immunotherapy based on chimeric antigen receptors or stem cell regeneration of differentiation based on artificial cells.

In some embodiments, the CISPA-linked effector protein, when reconstituted by ligand binding to the CISPA, is capable of regulating (promoting, suppressing, enhancing or inducing) an immune response. The term “immune response”, used herein, refers to any form of immune responses including antibody production (humoral response), induction of cell-mediated immunity (cellular cytotoxicity), complement activation and other. For example immune responses include the activation of cytokine responsive cells e.g. macrophages and T cells.

In some embodiments, CISPA is linked to a chimeric antigen receptor (CAR) as effector protein in a CAR-T cell therapy. CAR-T therapies are known in the art, and utilise T-cells that have been genetically engineered to produce an artificial T-cell receptor for use in immunotherapy. Chimeric antigen receptors are receptor proteins that have been engineered to give T-cells the new ability to target a specific protein, for example a tumour protein, pathogenic surface antigen, or other disease-related protein. T-cells for use in such a method may be derived from the patient to be treated, in which case the cells obtained from the patient are modified to express CISPA-CAR, optionally expanded, and administered to the patient. Alternatively, the T-cells may be derived from a (optionally tissue matched) donor, in which case the cells may be further modified to decrease their probability of rejection by the host body, or of attacking the host (as in graft versus host disease).

The patient to be treated may be any animal or human. The patient is preferably a non-human mammal, more preferably a human patient. The patient may be male or female.

Cells transformed with CISPA may be formulated as pharmaceutical compositions for clinical use and may comprise a pharmaceutically acceptable carrier, diluent or adjuvant. The composition may be formulated for topical, parenteral, intravenous, intramuscular, intrathecal, intraocular, subcutaneous, oral, inhalational or transdermal routes of administration which may include injection. Injectable formulations may comprise the selected compound in a sterile or isotonic medium.

Pharmaceutical compositions may be prepared using a pharmaceutically acceptable “carrier” composed of materials that are considered safe and effective. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 204(s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

The term “carrier” refers to diluents, binders, lubricants and disintegrants. Those with skill in the art are familiar with such pharmaceutical carriers and methods of compounding pharmaceutical compositions using such carriers.

### MISC. OTHER EMBODIMENTS/ASPECTS

Also disclosed herein is a kit comprising the CISPA split fragments forming a CISPA as described herein, optionally including the ligand. The kit may be for sensing a ligand, or may be for a therapeutic use, as described herein.

The disclosure also provides individual split fragments for a CISPA as described herein, in isolation or in the absence of the remaining fragments required to form a CISPA. Therefore, disclosed herein is a CISPA split fragment corresponding to the smaller or larger fragment of any CISPA described herein. The fragments may be joined to a section of an effector protein, as described herein. Exemplary CISPA fragments may comprise amino acids 1-179 of SEQ ID NO: 2, amino acids 515-585 of SEQ ID NO: 4, amino acids 1-187 of SEQ ID NO: 6, amino acids 515-569 of SEQ ID NO: 8, amino acids 1-214 of SEQ ID NO: 10, amino acids 515-562 of SEQ ID NO: 12, amino acids 1-231 of SEQ ID NO: 14, amino acids 515-562 of SEQ ID NO: 16, amino acids 1-30 of SEQ ID NO: 18, amino acids 515-759 of SEQ ID NO: 20, amino acids 1-174 of SEQ ID NO: 22, amino acids 515-527 of SEQ ID NO: 24, or a polypeptide having at least 80%, at least 90%, at least 95% or 100% identical amino acid residues thereof.

Also disclosed herein is a nucleic acid encoding any of the CISPA split fragments disclosed herein, a vector comprising said nucleic acid, and a cell comprising said nucleic acid or vector.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps. The word “consist”, and variations such as “consists” and “consisting”, will be understood to imply the inclusion of a stated integer or step or group of integers or steps and the exclusion of any other integer or step or group of integers or steps. Any use of the term “comprise”, “comprises” or “comprising” may be substituted for “consist”, “consists” or “consisting”, unless the context requires otherwise.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about.” it will be understood that the particular value forms another embodiment. The term “about” in relation to a numerical value is optional and means for example+/−10%.

### EXAMPLES

The examples that will be described in further detail have no intention of limiting the scope of the invention and its applicability, but are merely intended to provide a better understanding of the invention and its applicability.

### Example 1: Design and Preparation of DNA Constructs for Demonstration of the Invention

DNA constructs were prepared using methods of molecular biology that are described in any molecular biology handbook and are known to experts. In order to prepare DNA constructs, the inventors used experimental techniques and methods such as: chemical synthesis of DNA with a defined polynucleotide sequence, DNA fragmentation with restriction enzymes, DNA amplification using polymerase chain reaction-PCR, PCR ligation, DNA concentration determination, agarose gel electrophoresis, purification of DNA fragments from agarose gels, ligation of DNA fragments into a vector, the Gibson assembly method, transformation of chemically competent cells E. coli DH5a, isolation of plasmid DNA with commercially available kits, screening and selection. DNA fragments were characterized by restriction analysis and sequencing.

All plasmids, completed constructs and partial constructs were transformed into bacterium E. coli DH5alfa by chemical transformation. Plasmids for transfection into the cell line HEK293T were isolated using GeneJet Plasmid Miniprep DNA Isolation Kit (Thermo Fisher Scientific).

### Results:

All protein coding constructs have a Kozak sequence (GCCACC) before the coding region and were cloned into the pcDNA3 backbone vector for high-level expression in mammalian cells. In the described examples, the CISPAs are based on split ligand binding domain (LBD) of nuclear receptor superfamily (NRs) members, specifically ligand binding domains (LBDs) of glucocorticoid receptor (GR2), estrogen receptor beta (ERβ), thyroid receptor beta (TRβ), peroxisome proliferator-activated receptor gamma (PPARγ), human dihydrofolate reductase (DHFR) and kinase domain of tyrosine protein kinase Lyn. Split site position (S) for each protein or protein domain was chosen and the resulting fragments were cloned in such a way that, a starting fragment is cloned from the N-terminus of the protein to S and a terminating fragment from S+1 to the C-terminus of the protein.

Split site position is selected using known 3D structure or a molecular model of a protein-ligand complex in such a way that the size of the resulting split protein fragments are substantially different, with smaller fragment (nSplit or cSplit) comprising one to three segments of the secondary structure and the larger fragment (nSplit or cSplit) comprising the rest of the protein domain, with more amino acid residues than the smaller fragment. The majority of interactions, preferably 70% or more of the contacts of the cognate ligand with the selected protein, are located within the larger fragment of the split protein. Furthermore, split position is selected within the protein segment that is exposed to the solvent, preferably between polar amino acid residues, and is within the loop that connects protein secondary structure elements which are predicted to be more flexible and has lower amino acid residue conservation.

The DNA fragments encoding for the two split protein fragments were in-frame inserted into vectors containing sequences to code for linkers composed preferably from flexible glycine and serine rich amino acid residues, protein detection tags, nuclear localization signals, DNA-binding domains, transcriptional activation domains and split luciferase reporter fragments (nLuc and cLuc). All protein-coding DNA construct demonstrated as examples are listed in Table 1.

Constructs coding for guide RNAs are listed in Table 2 and were cloned into the pgRNA-humanized plasmid directly downstream of a murine U6 promoter which ensures high level expression of short RNAs in mammalian cells.

DNA target sequences are listed in Table 3 and were cloned into the pGL4.16 vector for expression in mammalian cells upstream of the minimal promoter (pMin).

TABLE 1
Composition of plasmids, used for
the demonstration of the invention

| Seq | Plasmid | Construct | |
|---|---|---|---|
| ID No. | name | composition | Vector |
| 1, 2 | nGR2:cLuc | nGR2:gs10:cLuc:gsg:HA | pcDNA3 |
| 3, 4 | nLuc:cGR2 | Myc:gsg:nLuc:gs10:cGR2 | pcDNA3 |
| 5, 6 | nERβ:cLuc | nERβ:gs10:cLuc:gsg:HA | pcDNA3 |
| 7, 8 | nLuc:cERβ | Myc:gsg:nLuc:gs10:cERβ | pcDNA3 |
| 9, 10 | nTRβ:cLuc | nTRβ:gs10:cLuc:gsg:HA | pcDNA3 |
| 11, 12 | nLuc:cTRβ | Myc:gsg:nLuc:gs10:cTRβ | pcDNA3 |
| 13, 14 | nPPARγ:cLuc | nPPARγ:gs10:cLuc:gsg:HA | pcDNA3 |
| 15, 16 | nLuc:cPPARγ | Myc:gsg:nLuc:gs10:cPPARγ | pcDNA3 |
| 17, 18 | nLyn:cLuc | nLyn:gs10:cLuc:gsg:HA | pcDNA3 |
| 19, 20 | nLuc:cLyn | Myc:gsg:nLuc:gs10:cLyn | pcDNA3 |
| 21, 22 | nDHFR:cLuc | nDHFR:gs10:cLuc:gsg:HA | pcDNA3 |
| 23, 24 | nLuc:cDHFR | Myc:gsg:nLuc:gs10:cDHFR | pcDNA3 |
| 25, 26 | nGR2:VPR | nGR2:gs10:NLS:VPR:gsg:Au1 | pcDNA3 |
| 27, 28 | dCas9:cGR2 | His:dCas9:NLS:gs10:cGR2 | pcDNA3 |
| 29, 30 | nERβ:VPR | nERβ:gs10:NLS:VPR:gsg:Au1 | pcDNA3 |
| 31, 32 | dCas9:cERβ | His:dCas9:NLS:gs10:cERβ | pcDNA3 |
| 33, 34 | nTRβ:VPR | nTRβ:gs10:NLS:VPR:gsg:Au1 | pcDNA3 |
| 35, 36 | dCas9:cTRβ | His:dCas9:NLS:gs10:cTRβ | pcDNA3 |
| 37, 38 | nPPARγ:VPR | nPPARγ:gs10:NLS:VPR:gsg:Au1 | pcDNA3 |
| 39, 40 | dCas9:cPPARγ | His:dCas9:NLS:gs10:cPPARγ | pcDNA3 |
| 41, 42 | nLyn:VPR | nLyn:gs10:NLS:VPR:gsg:Au1 | pcDNA3 |
| 43, 44 | dCas9:cLyn | His:dCas9:NLS:gs10:cLyn | pcDNA3 |
| 45, 46 | nDHFR:VPR | nDHFR:gs10:NLS:VPR:gsg:Au1 | pcDNA3 |
| 47, 48 | dCas9:cDHFR | His:dCas9:NLS:gs10:cDHFR | pcDNA3 |

TABLE 2
Guide RNAs, used for the demonstration of the invention

| Seq | Guide | | |
|---|---|---|---|
| ID No. | sequence | Description | Vector |
| 49 | [AB]nt | The gRNA targets a sequence | pgRNA- |
| | | designated [AB], non- | humanized |
| | | template strand | |

TABLE 3
DNA target sequences, used for the demonstration of the invention

| Seq | DNA target | Reporter | | |
|---|---|---|---|---|
| ID No. | sequence | plasmid | Description | Vector |
| 50 | [AB] | 10[AB]_Pmin_fLuc | The plasmid contains 10 copies of target | pGL4.16 |
| | | | sequence designated [AB] | |

### Example 2: Demonstration of Ligand Mediated CISPA Sensing Based on Split Ligand Binding Domain (LBDs) of Nuclear Receptor Superfamily (NRs) Members

Herein, the inventors demonstrate that split fragments (nSplit and cSplit) based on the ligand binding domains (LBDs) of nuclear receptor superfamily (NRs) members, dimerize in the presence of a cognate ligand. In particular, the inventors have demonstrated herein, that CISPAs based on split LBDs of NRs, including but not limited, to glucocorticoid receptor (GR2), thyroid receptor beta (TRβ), peroxisome proliferator-activated receptor gamma (PPARγ) and estrogen receptor beta (ERβ), can be used for sensing of their cognate ligands in mammalian cells.

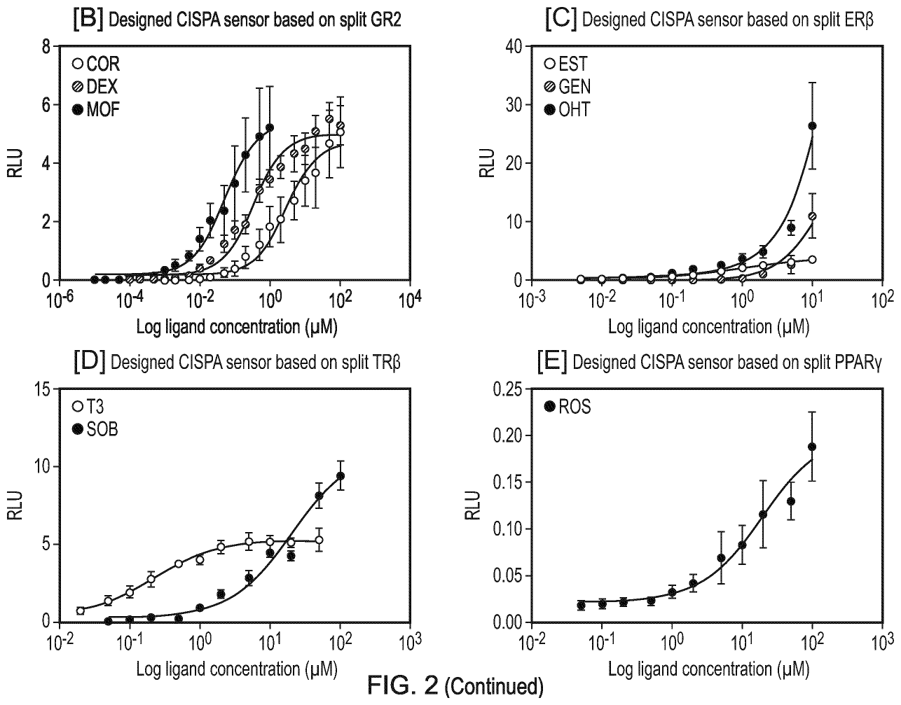

To demonstrate the use of CISPAs, for in situ sensing of ligands, the inventors genetically fused N-terminal and C-terminal fragments of selected split LBDs to a well know split firefly luciferase reporter. The fusion constructs were simultaneously expressed in the HEK293T cell line. As exemplified in FIG. 2A when a specific cognate ligand is present, the split fragments of CISPA sensor reassemble. Following this dimerization, the split reporter fragments (nLuc and cLuc) are brought into the proximity, leading to their reconstitution and measurable output signal (emitted light). The plasmids encoding for the CISPA sensors based on split LBDs were transiently transfected into the HEK293T cell line. A constitutively expressed Renilla luciferase (phRL-TK http://www.promega.com/vectors/prltk.txf) was used as control of transfection efficiency.

### Cell Culture, Transfection and Stimulation

Methods and techniques for cell line cultivation are well-known to experts in the field and are explained here only indicatively, with the intention of clarifying the example. Cell cultures of HEK293T cells were grown at 37° C. and 5% CO₂. For cultivation, DMEM medium with 10% FBS, containing all the necessary nutrients and growth factors was used. When the cell population reached a sufficient density, the cells were transferred to a new flask and/or diluted. For use in experiments, the number of cells was determined by the Countess automated cell counter (Invitrogen). A 96-well microtiter plate, suitable for growing cell cultures, was inoculated with 2*10⁴ cells per well 24 hours prior to transfection. Inoculated plates were incubated at 37° C. and 5% CO₂. At 30-90% confluence, they were transfected with a mixture of DNA and PEI (6 μl/500 ng DNA, stock concentration 0.324 mg/ml, pH 7.5) and further incubated at 37° C. and 5% CO₂. Twelve hours after transfection, the cells were stimulated by removing the media and replacing it with media containing selected ligands. Cells were stimulated for 24 hours.

### Ligand Preparation and Sources

A 1000× stock solutions of cortisol (100 mM, Sigma Aldrich, Catalog No. H-0888), dexamethasone (100 mM, Sigma Aldrich, Catalog No. D-1756), mometasone furoate (10 mM, Sigma Aldrich, Catalog No. M4074), 17β-estradiol (10 mM, Sigma Aldrich, Catalog No. E8875), 4-hydroxytamoxifen (10 mM, Sigma Aldrich, Catalog No. H6278), genistein (10 mM, Sigma Aldrich, Catalog No. G-6649), triiodothyronine (50 mM, Sigma Aldrich, Catalog No. T2877), sobetirome (100 mM, Sigma Aldrich, Catalog No. SML1900), rosiglizatone (100 mM, Sigma Aldrich, Catalog No. R2408), dasatinib (10 mM, AdooQ Bioscience, Catalog No. A10290-25), methotrexate (1 mM, Sigma Aldrich, Catalog No. A6770) and pralatrexate (1 mM, Sigma Aldrich, Catalog No. SML2494) were prepared in 100% DMSO and stored at −20° C. Each of the selected ligands was added to cell cultures such that the final concentration was 1× at the time of stimulation.

### Dual Luciferase Assay

Two days after transfection, the cells were harvested and lysed with 25 μl of 1× passive lysis buffer (Promega). Firefly luciferase and Renilla luciferase expression were measured using a dual luciferase assay (Promega) and an Orion II microplate reader (Berthold Technologies). The dual luciferase method used is described in the manufacturer's instructions (Promega). rLuc activity indicates the proportion of successfully transfected cells, while fLuc activity shows activation of reporter gene expression. Relative luciferase activity (RLU) was calculated by normalizing each sample's firefly luciferase activity to the constitutive Renilla luciferase activity determined within the same sample.

### Results:

The results shown in FIG. 2B demonstrate ligand-mediated dimerization of CISPA sensor based on split LBDs of NR members, specifically split ligand binding domain of GR2, fused to a split luciferase reporter. Upon stimulation of HEK293T cells with increasing concentration of cortisol (COR), an increase in the firefly luciferase activity is observed compared to the cells that are not stimulated. Furthermore, we demonstrate that CISPA sensor based on split LBD of GR2 can be used to detect synthetic glucocorticoids such as mometasone furoate (MOF) and dexamethasone (DEX) also in a concentration-dependent manner.

The results shown in FIG. 2C demonstrate the ligand-mediated dimerization of CISPA sensor based on split LBDs of NRs members, specifically split ligand binding domain of ERP, fused to split luciferase reporter. Upon stimulation of HEK293T cells with increasing concentration of 17β-estradiol (EST), an increase in firefly luciferase activity is observed compared to the cells that are not stimulated. This confirms that our CISPA can be used for in vivo sensing of 17β-estradiol in a concentration dependent manner. Additionally, we demonstrate that CISPA sensor based on split LBD of ERP can be used to detect non-steroidal selective estrogen receptor modulator 4-hydroxytamoxifen (OHT) and a phytoestrogen genistein (GEN) also in concentration-dependent manner.

The results shown in FIG. 2D demonstrate the ligand-mediated dimerization of CISPA sensor based on split LBDs of NR members, specifically split ligand binding domain of TRP, fused to the split luciferase reporter. Upon stimulation of HEK293T cells with increasing concentration of triiodothyronine (T3), an increase in firefly luciferase activity is observed in compared to the cells that are not stimulated. Furthermore, we demonstrate that our CISPA sensor based on split LBD of TRβ can be used to detect selective TRβ analog sobetirome (SOB) also in a concentration dependent manner.

The results shown in FIG. 2E demonstrate the rosiglitazone (ROS) mediated dimerization of CISPA sensor based on split ligand binding domain of PPARγ, fused to split luciferase reporter. Upon stimulation of HEK293T cells with increasing concentration of rosiglizatione, an increase in firefly luciferase activity can be observed in comparison with the cells that were not stimulated.

The results as shown herein confirm that our CISPA sensors based on split LBDs, including but not limited, to LBDs of glucocorticoid receptor (GR2), estrogen receptor beta (ERβ), thyroid receptor beta (TRβ) and peroxisome proliferator-activated receptor gamma (PPARγ) can be used for sensing of their cognate ligands in concentration dependent manner.

### Example 3: Demonstration of Ligand Mediated CISPA Sensing Based on Split Kinase Domain of Tyrosine Protein Kinase Lyn

The CISPA sensor based on split Lyn kinase domain was constructed based on the principles as described above. Briefly, nLyn and cLyn fragments were genetically fused to split firefly luciferase reporter. In the presence of a ligand inhibitor dasatinib the split fragments of CISPA reassemble, resulting in reconstitution of split luciferase and emitted light.

The plasmids encoding for designed CISPA sensor were transiently co-transfected into HEK293T cell line. A constitutively expressed Renilla luciferase (phRL-TK http://www.promega.com/vectors/prltk.txf) was used as control of transfection efficiency.

The HEK293T cells were cultured, transfected and stimulated as described above.

### Results:

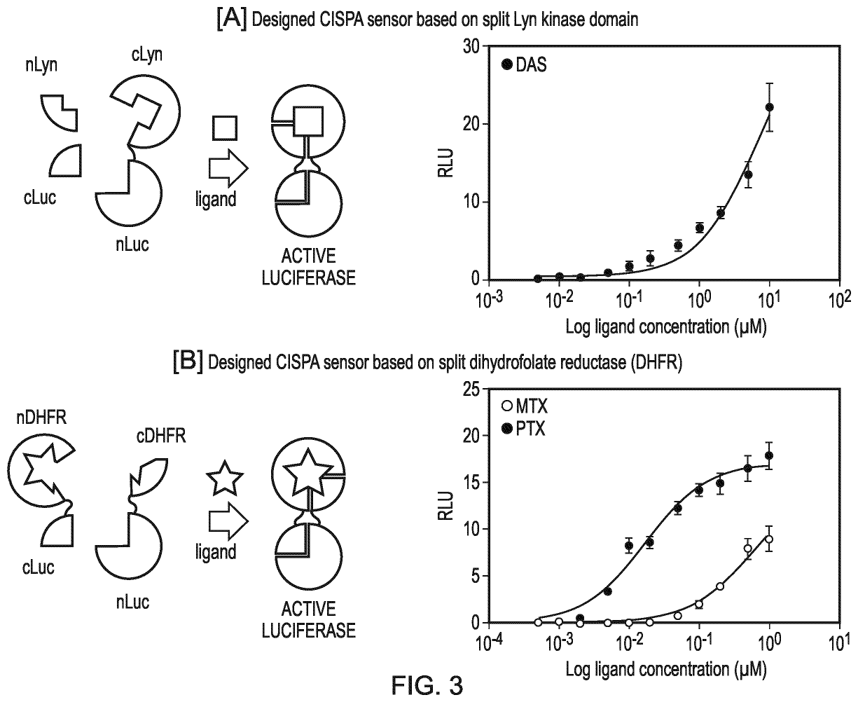

The results presented in FIG. 3A demonstrate the use of CISPA for in situ sensing of ligand inhibitor dasatinib (DAS). Upon stimulation of HEK293T cells with increasing concentration of ligand dasatinib, an increase in firefly luciferase activity is observed in comparison with the cells that are not stimulated.

The results as shown herein, confirm that our CISPA sensor based on split kinase domain of tyrosine protein kinase Lyn can be used for sensing of ligand inhibitor dasatinib in a concentration dependent manner.

### Example 4: Demonstration of Ligand Mediated CISPA Sensing Based on Split Dihydrofolate Reductase (DHFR)

The CISPA sensor based on split dihydrofolate reductase (DHFR) was constructed based on principles as described above. nDHFR and cDHFR fragments were genetically fused to a split firefly luciferase reporter. In the presence of a ligand inhibitor the split fragments of CISPA reassemble, resulting in reconstitution of split luciferase and emitted light.

The plasmids encoding for CISPA sensor was transiently co-transfected into HEK293T cell line. A constitutively expressed Renilla luciferase (phRL-TK http://www.promega.com/vectors/prltk.txf) was used as control of transfection efficiency.

The HEK293T cells were cultured, transfected and stimulated as described above.

### Results:

The results presented in FIG. 3B demonstrate the use of CISPA for in situ sensing of methotrexate (MTX) or pralatrexate (PTX). Upon stimulation of HEK293T cells with increasing concentration of methotrexate or pralatrexate, an increase in firefly luciferase activity is observed in comparison with the cells that were not stimulated. The results as shown herein, confirm that our CISPA sensor based on dihydrofolate reductase (DHFR) can be used for in situ sensing of ligand inhibitors methotrexate and pralatrexate in concentration dependent manner.

### Example 5: Demonstration of CISPA Systems for Ligand-Mediated Control of Transcriptional Activation

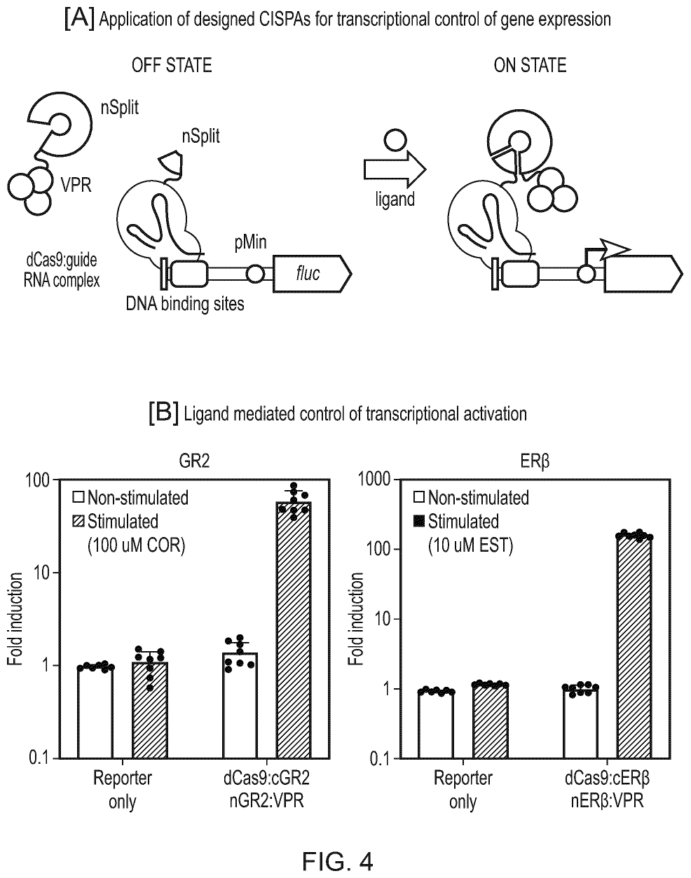

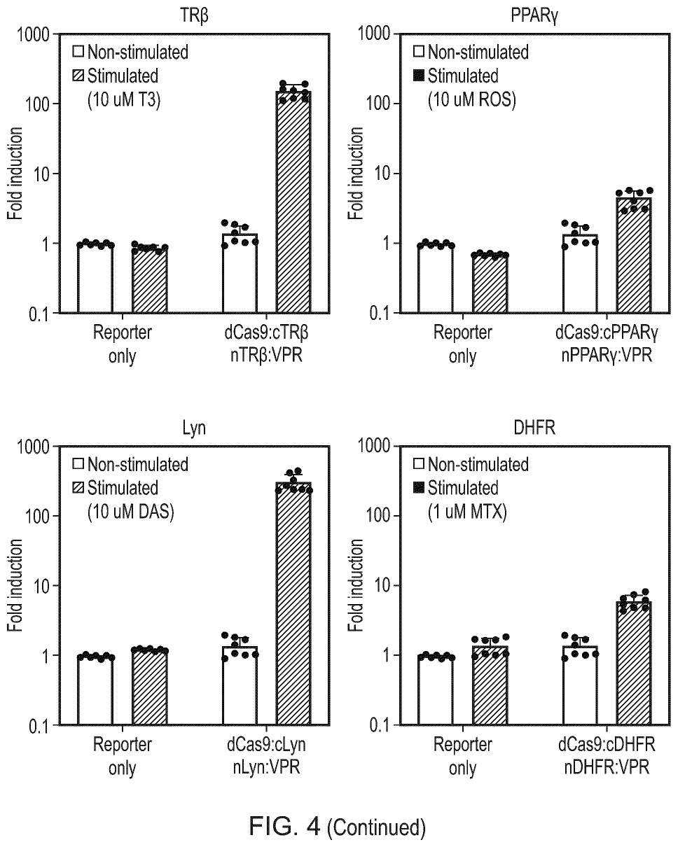

In one embodiment of the invention, CISPAs can be used for control of cellular processes such protein localization, protein stability and signal transduction. To show the applicability of our invention to control cellular processes we demonstrate the use of CISPAs for the cognate ligand control of transcriptional activation. For this purpose we fused the nSplit fragments of CISPAs to transcriptional activation domain VPR (nSplit:VPR encoding plasmids) and cSplit fragments to S. pyogenes catalytically inactive Cas9 (dCas9) DNA-binding domain (dCas9:cSplit encoding plasmids). When the selected ligand is present, it will cause the dimerization of split protein fragments and recruitment of the transcriptional activation domain VPR into the close proximity of a minimal promoter (pMin), driving the expression of a firefly luciferase reporter gene (fLuc) (FIG. 4A).

The plasmids encoding the reporter plasmids containing the dCas9:guide RNA DNA target sequences, VPR transcriptional activation domains and dCas9 DNA-binding domains fused to the appropriate designed split CISPA fragments (nSplit and cSplit) were transiently co-transfected into the HEK293T cell line. A constitutively expressed Renilla luciferase (phRL-TK http://www.promega.com/vectors/prltk.txf was used as controls of transfection efficiency. The HEK293T cells were cultured, transfected and stimulated as described above.

### Results:

The results shown in FIG. 4B demonstrate the ligand-mediated activation of reporter gene expression. From the results it is evident, that ligand alone does not activate the transcription of the reporter gene, if cells are transfected with only the reporter plasmid encoding for 10 binding sites for designed transcription factor dCas9:guide complex. Ligand-mediated activation of reporter gene is observed only in case when HEK293T cells, co-transfected with plasmids encoding for designed split CISPAs fused to DNA-binding domain dCas9 (dCas9:cSplit) and transcriptional activation domain VPR (nSplit:VPR), were stimulated with cognate ligands. Therefore, the results as shown herein, confirm that our CISPAs based on split ligand binding domains (LBDs) of glucocorticoid receptor (GR2), estrogen receptor beta (ERβ), thyroid receptor beta (TRβ), peroxisome proliferator-activated receptor gamma (PPARγ), kinase domain of tyrosine protein kinase Lyn and dihydrofolate reductase (DHFR) can be used for ligand-mediated activation of the reporter gene. Furthermore, similar results could be achieved using other DNA-binding domains, such as zinc fingers, TALEs and other specific DNA binding domains combined with transcriptional activation domains or transcriptional repression domains.

### REFERENCES

A number of publications are cited above in order to more fully describe and disclose the invention and the state of the art to which the invention pertains. Full citations for these references are provided below.

The entirety of each of these references is incorporated herein.
Derose, R., Miyamoto, T., & Inoue, T. (2013). Manipulating signaling at will: Chemically-inducible dimerization (CID) techniques resolve problems in cell biology. Pflugers Archiv European Journal of Physiology. https://doi.org/10.1007/s00424-012-1208-6
Duong, M. L. T., Collinson-Pautz, M. R., Morschl, E., Lu, A., Szymanski, S. P., Zhang, M., Bayle, J. H. (2019). Two-Dimensional Regulation of CAR-T Cell Therapy with Orthogonal Switches. Molecular Therapy—Oncolytics, 12, 124-137. https://doi.org/10.1016/j.omto.2018.12.009
Fink, T., Lonzarić, J., Praznik, A., Plaper, T., Merljak, E., Leben, K., Jerala, R. (2019). Design of fast proteolysis-based signaling and logic circuits in mammalian cells. Nature Chemical Biology, 15(2), 115-122. https://doi.org/10.1038/s41589-018-0181-6
Foight, G. W., Wang, Z., Wei, C. T., Greisen, P., Warner, K. M., Cunningham-Bryant, D., Maly, D. J. (2019). Multi-input chemical control of protein dimerization for programming graded cellular responses. Nature Biotechnology, 37(10), 1209-1216. https://doi.org/10.1038/s41587-019-0242-8
Gao, Y., Xiong, X., Wong, S., Charles, E. J., Lim, W. A., & Qi, L. S. (2016). Complex transcriptional modulation with orthogonal and inducible dCas9 regulators. Nature Methods, 13(12), 1043-1049. https://doi.org/10.1038/nmeth.4042
Glasgow, A. A., Huang, Y. M., Mandell, D. J., Thompson, M., Ritterson, R., Loshbaugh, A. L., Kortemme, T. (2019). Computational design of a modular protein sense-response system. Science, 366(6468), 1024-1028. https://doi.org/10.1126/science.aax8780
Hill, Z. B., Martinko, A. J., Nguyen, D. P., & Wells, J. A. (2018). Human antibody-based chemically induced dimerizers for cell therapeutic applications. Nature Chemical Biology, 14(2), 112-117. https://doi.org/10.1038/nchembio.2529
Kang, S., Davidsen, K., Gomez-Castillo, L., Jiang, H., Fu, X., Li, Z., Gu, L. (2019). COMBINES-CID: An Efficient Method for de Novo Engineering of Highly Specific Chemically Induced Protein Dimerization Systems. Journal of the American Chemical Society, 141(28), 10948-10952. https://doi.org/10.1021/jacs.9b03522
Miyamoto, T., DeRose, R., Suarez, A., Ueno, T., Chen, M., Sun, T., Inoue, T. (2012). Rapid and orthogonal logic gating with a gibberellin-induced dimerization system. Nature Chemical Biology, 8(5), 465-470. https://doi.org/10.1038/nchembio.922
Pissios, P., Tzameli, I., Kushner, P. J., & Moore, D. D. (2000). Dynamic stabilization of nuclear receptor ligand binding domains by hormone or corepressor binding. Molecular Cell, 6(2), 245-253. https://doi.org/10.1016/S1097-2765(00)00026-5
Rivera, V. M., Clackson, T., Natesan, S., Pollock, R., Amara, J. F., Keenan, T., Gilman, M. (1996). A humanized system for pharmacologic control of gene expression. Nature Medicine, 2(9), 1028-1032. https://doi.org/10.1038/nm0996-1028
Schellekens, H. (2005). Factors influencing the immunogenicity of therapeutic proteins. Nephrology Dialysis Transplantation, 20(suppl_6), vi3-vi9. https://doi.org/10.1093/ndt/gfh1092
Stanton, B. Z., Chory, E. J., & Crabtree, G. R. (2018). Chemically induced proximity in biology and medicine. Science (New York, N.Y.), 359(6380). https://doi.org/10.1126/science.aao5902 Wu, C.-Y., Roybal, K. T., Puchner, E. M., Onuffer, J., & Lim, W. A. (2015). Remote control of therapeutic T cells through a small molecule-gated chimeric receptor. Science, 350(6258), aab4077-aab4077. https://doi.org/10.1126/science.aab4077
Tanenbaum M E, Gilbert L A, Qi L S, Weissman J S, Vale R D. A protein-tagging system for signal amplification in gene expression and fluorescence imaging. Cell. 2014 Oct. 23; 159(3):635-46. doi: 10.1016/j.cell.2014.09.039. Epub 2014 Oct. 9. PMID: 25307933; PMCID: PMC4252608.
Ye, X., Rivera, V. M., Zoltick, P., Cerasoli, F., Schnell, M. A., Gao, G. P., Wilson, J. M. (1999). Regulated delivery of therapeutic proteins after in vivo somatic cell gene transfer. Science, 283(5398), 88-91. https://doi.org/10.1126/science.283.5398.88
Zhao, W., Nguyen, H., Zeng, G., Gao, D., Yan, H., & Liang, F.-S. (2018). A chemically induced proximity system engineered from the plant auxin signaling pathway. Chemical Science, 9(26), 5822-5827. https://doi.org/10.1039/C8SC02353K
For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A method of designing a chemically inducible split protein actuator (CISPA), wherein the CISPA comprises two split fragments capable of forming a heterodimer in the presence of a ligand, the method comprising:
i. selecting a ligand-binding protein or protein domain, wherein the ligand-binding protein or protein domain is capable of binding a ligand,
ii. providing a 3D structure or molecular model of the ligand-binding protein or protein domain in complex with the ligand, and
iii. selecting a split site position within said 3D structure or model so as to divide the ligand-binding protein or protein domain into two split fragments,
wherein the two split fragments are unequal in size, the smaller split fragment comprises no more than one third of the ligand-binding protein or protein domain, the larger split fragment comprises the remainder of the ligand-binding protein or protein domain, and wherein the majority of ligand interactions contact amino acid residues within the larger split fragment.

2. A chemically inducible split protein actuator (CISPA) comprising two split fragments capable of forming a heterodimeric ligand-binding protein or protein domain in the presence of a ligand,
wherein
the two split fragments are unequal in size, and
the smaller split fragment comprises no more than one third of the ligand-binding protein or protein domain, the larger split fragment comprises the remainder of the ligand-binding protein or protein domain, and
the majority of ligand interactions contact amino acid residues within the larger split fragment.

3. The method or CIPSA according to claim 1 or claim 2, wherein the smaller split fragment contains between one and three segments of alpha helices or beta strands.

4. The method or CIPSA according to any previous claim, wherein at least 70% interactions between the ligand-binding protein or domain and the ligand contact amino acid residues within the larger split fragment.

5. The method or CIPSA according to any previous claim, wherein the split fragments are fused to a first and a second segment of an effector protein or protein domain such that the function of the effector protein or protein domain is reconstituted when the heterodimer is formed in the presence of the ligand.

6. The method or CIPSA according to claim 5, wherein the effector protein or protein domain is a reporter which, when reconstituted, generates a detectable chemical or physical signal.

7. The method or CIPSA according to claim 5, wherein the reporter is selected from a split luciferase, fluorescent protein, phosphatase, protease, or oxidoreductase.

8. The method or CIPSA according to claim 5, wherein the effector protein or protein domain is a split protease, localization signal, DNA- or RNA-binding domain, recombinase, transcriptional regulator, or chromatin-remodelling domain, or a combination thereof.

9. The method or CIPSA according to claim 5, wherein the effector protein or protein domain is a transcriptional regulator.

10. The method or CIPSA according to claim 9, wherein the first segment of the transcriptional regulator is a DNA-binding domain and the second segment of the transcriptional regulator is selected from a transcriptional activation domain and a transcriptional repression domain, or vice-versa.

11. The method or CIPSA according to claim 10, wherein the DNA-binding domain is a catalytically inactive Cas9 (dCas9), and the transcriptional activation domain is VP64-p65-Rta (VPR).

12. The method or CIPSA according to any previous claim, wherein the ligand-binding protein or protein domain is a human ligand-binding protein or protein domain.

13. The method or CIPSA according to any previous claim, wherein the ligand-binding protein or protein domain is a nuclear receptor (NR) superfamily member, a Src family protein tyrosine kinase, dihydrofolate reductase (DHFR), or a fragment thereof.

14. The method or CIPSA according to any previous claim, wherein the ligand-binding protein or protein domain is glucocorticoid receptor (GR), thyroid receptor beta (TRβ), peroxisome proliferator-activated receptor gamma (PPARγ) or estrogen receptor beta (ERβ), dihydrofolate reductase (DHFR), tyrosine protein kinase Lyn, tyrosine protein kinase Lck, tyrosine protein kinase Yes, tyrosine protein kinase Fyn, or a fragment thereof.

15. The method or CIPSA according to any previous claim, wherein the ligand is a human protein or fragment thereof, or a pharmacological compound.

16. The method or CIPSA according to any previous claim, wherein the ligand has a molecular weight of 5 kDa or less.

17. The method or CIPSA according to any of claims 1 to 11, wherein:
i. the smaller split fragment comprises amino acids 515-585 of SEQ ID NO:4 or a polypeptide having at least 80% identical amino acid residues thereof, the second split fragment comprises amino acids 1-179 of SEQ ID NO:2 or a polypeptide having at least 80% identical amino acid residues thereof, and the ligand is capable of binding a glucocorticoid receptor (GR);
ii. the smaller split fragment comprises amino acids 515-569 of SEQ ID NO:8 or a polypeptide having at least 80% identical amino acid residues thereof, the larger split fragment comprises amino acids 1-187 of SEQ ID NO:6 or a polypeptide having at least 80% identical amino acid residues thereof, and the ligand is capable of binding to ERP;
iii. the smaller split fragment comprises amino acids 515-562 of SEQ ID NO:12 or a polypeptide having at least 80% identical amino acid residues thereof, the larger split fragment comprises amino acids 1-214 of SEQ ID NO:10 or a polypeptide having at least 80% identical amino acid residues thereof, and the ligand is capable of binding to tRP;
iv. the smaller split fragment comprises amino acids 515-562 of SEQ ID NO:16 or a polypeptide having at least 80% identical amino acid residues thereof, the larger split fragment comprises amino acids 1-231 of SEQ ID NO:14 or a polypeptide having at least 80% identical amino acid residues thereof, and the ligand is capable of binding to PPARγ;
v. the smaller split fragment comprises amino acids 1-30 of SEQ ID NO:18 or a polypeptide having at least 80% identical amino acid residues thereof, the larger split fragment comprises amino acids 515-759 of SEQ ID NO:20 or a polypeptide having at least 80% identical amino acid residues thereof, and the ligand is capable of binding to Lyn; or
vi. the smaller split fragment comprises amino acids 515-527 of SEQ ID NO:24 or a polypeptide having at least 80% identical amino acid residues thereof, the larger split fragment comprises amino acids 1-174 of SEQ ID NO:22 or a polypeptide having at least 80% identical amino acid residues thereof, and the ligand is capable of binding to DHFR.

18. A method of producing a chemically inducible split protein actuator (CISPA), comprising the steps of:
i. designing a CISPA according to any previous claim, and
ii. producing the split fragments according to the design.

19. A nucleic acid or set of nucleic acids encoding a chemically inducible split protein actuator (CISPA) according to any one of claims 1 to 17.

20. A vector or set of vectors encoding the nucleic acid or acids of claim 19.

21. A cell comprising the nucleic acid or acids of claim 19, or the vector or vectors of claim 20.

22. A method of detecting a ligand, comprising
i. contacting the ligand with a CISPA according to claim 6 or claim 7, and
ii. measuring the detectable chemical or physical signal produced by the reporter,
wherein the CISPA is capable of binding the ligand.

23. A method of regulating transcription of a gene, comprising:
contacting a nucleic acid encoding the gene with a CISPA according to any one of claims 9 to 11, and
contacting the CISPA with the ligand capable of binding the CISPA.

24. A method of regulating a cellular process, comprising:
introducing a CISPA according to claim 8 into a cell, and
contacting the CISPA with the ligand capable of binding the CISPA,
wherein the cellular process is regulated by the effector protein or protein domain.

25. A method of treatment comprising:
i. administering a therapeutic cell comprising or capable of expressing a CISPA according to claim 5, to a patient in need thereof, wherein the effector protein or protein domain regulates a therapeutic process, and
ii. contacting the therapeutic cell with the CISPA ligand.

26. A method of treatment comprising:
i. providing a cell derived from a patient in need of cell therapy,
ii. modifying the cell to express a CISPA according to claim 5, wherein the effector protein or protein domain is capable of regulating a therapeutic process,
iii. administering the modified cell to the patient, and
iv. contacting the therapeutic cell with the CISPA ligand.

27. A therapeutic cell for use in a method of cell therapy, wherein the therapeutic cell comprises or is capable of expressing a CISPA according to claim 5 wherein the effector protein or protein domain regulates a therapeutic process.

28. Use of a therapeutic cell in the manufacture of a medicament for cell therapy, wherein the therapeutic cell comprises or is capable of expressing a CISPA according to claim 5 wherein the effector protein or protein domain regulates a therapeutic process.

29. The therapeutic cell for use according to claim 27 or the use according to claim 27, wherein the method comprises the step of contacting the therapeutic cell with the CISPA ligand.

30. The method, therapeutic cell for use, or use according to any one of claims 25 to 29, wherein the effector protein or protein domain is a chimeric antigen receptor.
